(19) United States
(12) Patent Application Publication
(10) Pub. No.: US 2024/0108759 A1
(43) Pub. Date: Apr. 4, 2024

(54) NUCLEIC ACID TRANSPORTERS IN NANOCHAIN FORM, PREPARATION METHOD THEREFOR, AND PHARMACEUTICAL COMPOSITION FOR CANCER GENE THERAPY, COMPRISING SAME

(71) Applicant: ELBIO INC, Seoul (KR)

(72) Inventors: Jong Hoon CHUNG, Seoul (KR); Pankaj GARG, Seoul (KR); Shambhavi PANDEY, Seoul (KR); Myung Chul LEE, Hanam-si, Gyeonggi-do (KR)

(73) Assignee: ELBIO INC, Seoul (KR)

(21) Appl. No.: 18/273,526

(22) PCT Filed: Jan. 24, 2022
(86) PCT No.: PCT/KR2022/001260

§ 371 Date: Jul. 20, 2023

(30) Foreign Application Priority Data
Jan. 25, 2021 (KR) 10-2021-0010461

(51) Int. Cl. A61K 48/00; A61K 47/54; A61P 35/00

(52) U.S. Cl. CPC A61K 48/0075; A61K 47/549; A61K 48/0041; A61P 35/00

(57) ABSTRACT

The present invention relates to a polydixylitol polymer nucleic acid transporter (X-NC) prepared in a nanochain form, a method for preparing the same, and a technology for treating brain tumors by using the same, a nucleic acid transporter complex in which a therapeutic nucleic acid is linked to the nucleic acid transporter, and a pharmaceutical composition for gene therapy, comprising the complex as an active ingredient.

### TECHNICAL FIELD

The present invention relates to a polydixylitol-based nanochain nucleic acid transporter (nanochain synthesized from polydixylitol/nucleic acid transporter, X-NC) in which polydixylitol-based genes, that is, nucleic acid transporter (polydixylitol polymer-based nucleic acid transporter, PdXYP, X-NP) are linked in a chain form, and a method for preparing thereof. In addition, the present invention relates to a nucleic acid transporter complex in which a therapeutic nucleic acid is conjugated to the nucleic acid transporter and a pharmaceutical composition for gene therapy comprising the complex as an active ingredient. In addition, it relates to the treatment of cancer such as brain tumor using the gene transfer complex.

### BACKGROUND ART

Nano pharmaceuticals designed to reach the central nervous system (CNS) must pass through the highly evolved microvessels of the blood-brain barrier (BBB) which prevents most therapeutic drugs from entering the brain. The BBB consists of neurovascular units connected by tight junctions and tightly regulates the movement of molecules between the blood and the brain. However, during tumor formation, this BBB loses its integrity and a highly permeable blood-tumor barrier (BTB) is formed. Despite the increased permeability of the BTB, it may be permeated heterogeneously, not conducive to the entry of therapeutic drugs into the interior of the brain tumor due to the efflux activity of the cells. Moreover, solid tumors have a poorly organized vasculature and increased interstitial fluid pressure that slows the movement of molecules, making anticancer drugs inaccessible to deeply located cells. The impermeability of various and highly effective treatment drugs for tumors into the brain precludes drug therapy or requires the use of invasive therapy, limiting their effectiveness. Therefore, for cancer treatment to be effective in the brain, a drug must penetrate deeper into the tumor stroma at optimal concentrations, cross the BBB and BTB, and retain pharmacological activity.

Numerous nanoparticles (NPs) of various shapes have been devised for gene therapy by targeting brain tumors beyond the BBB. However, the bioavailability of spherical nanoparticles is low because they are uneven in shape, most nanoparticles accumulate around blood vessels, and most of them do not exist in avascular regions of tumors while circulating in vivo. On the other hand, the non-spherical shape increases the transport probability along the bloodstream and improves the transport of particles due to reduced steric hindrance due to viscous drag force near the vessel wall. In addition, oblate-shaped particles with a high aspect ratio can easily avoid uptake by macrophages in the reticuloendothelial system, increasing their distribution in vivo. In addition, at the target site, non-spherical particles subjected to rotational force moved laterally toward the vessel wall and were deposited several times more than spherical particles.

The aspect ratio of non-spherical particles also determines the efflux rate and extent of intratumoral deposition, improving treatment efficiency. Chain-shaped nanochains composed of metal nanoparticles (e.g., iron oxide, gold) and drug-loaded liposomes have been studied for high frequency-induced drug release as chemotherapeutic drugs for brain tumors. Mechanisms that induce drug release according to different temperature and pH sensitivities have also been applied to nanoparticle systems. However, the drug release method that controls time and space has limitations on drug loading efficiency. It requires the construction of smart multi-component vectors that can not only cross the BBB and BTB, but also deliver the right amount of gene drug towards its target in avascular regions deep within the tumor.

Herein, the present inventors propose a key treatment strategy for diseases related to the central nervous system by solving the long-standing challenge of gene delivery across the BBB and BTB.

### DISCLOSURE

### Technical Problem

One object of the present invention is to provide a nucleic acid transporter that can pass through the BBB and BTB, and has significantly improved transfection efficiency without showing cytotoxicity.

Another object of the present invention is to provide a method for preparing the nucleic acid transporter in a nanochain form.

Still another object of the present invention is to provide a nucleic acid transporter complex in which a therapeutic nucleic acid is conjugated to the nucleic acid transporter in the nanochain form and a pharmaceutical composition comprising the same.

### Technical Solution

As one aspect for achieving the above object, a method of manufacturing previously invented polydixylitol polymer (PdXYP) (Chemical Formula I) in a chain form using dixylitol diacrylate (dXYdA), and a nucleic acid transporter in a nanochain form manufactured by the method are provided.

As an additional aspect, a gene transporter complex loaded with a therapeutic nucleic acid on the nucleic acid transporter in a nanochain form and a pharmaceutical composition using the same are provided.

### Advantageous Effects

It was confirmed that the nucleic acid transporter in a nanochain form of the present invention (X-NC) to which polydixylitol polymer (PdXYP) is linearly linked has a significantly higher nucleic acid delivery ratio to cancer cells than the existing nucleic acid transporters, and passes through the blood-brain barrier and transfers nucleic acid to cancer cells to transform them, and the mechanism thereof was identified. Accordingly, the nucleic acid transporter of the present invention is expected to be widely used in the field of gene therapy for various cancer diseases by suppressing the growth of tumors in vivo.

### DESCRIPTION OF DRAWINGS

FIGS. 1A-1I show the synthesis process and characterization analysis of X-NP/X-NC of the present invention.

FIG. 1A is a diagram of the synthesis steps of a polydixylitol-based nanochain (X-NC) that delivers nucleic acid. FIG. 1B shows a higher transformation (transfection) efficiency of X-NC than X-NP in the FACS data of A549 and GBM cells treated with the nanochain. FIG. 1C is the result of the hydrodynamic particle size among the DLS measurement results of X-NC/DNA, X-NP/DNA and PEI25k/DNA (N/P 10). FIG. 1D is the zeta potential measurement result, FIG. 1E shows the permeability of X-NC, X-NP, and PEI. FIG. 1F represents efficiency data versus % of GFP transfection efficiency measured by FACS (n=3, error bars indicate standard deviation) (***P<0.001, *P<0.05, one-way ANOVA). FIG. 1G is an EF-TEM image of (i) spherical X-NPs (scale: 100 nm) and (ii) linearly aligned X-NCs (scale: 500 nm). At this time, the average particle size was calculated using ImageJ software, and the average aspect ratio of X-NC was found to be 3:1 or less. FIG. 1H represents the transgenic GFP expression of human lung cancer cells (A549) (Scale: 200 μm). FIG. 1I is a reverse contrast image of directional X-NC/DNA^{YOYO}(brightly illuminated area indicated by an arrow) introduced into cells 60 min after transfection of A549 cells (Scale: 100 μm).

FIGS. 2A-2D show that high osmotic pressure induces the expression of NFAT5 and the entry of X-NC into cells. FIG. 2A is an illustration of BBB permeation by the osmotic stress inducing mechanism of X-NC and cell influx through NFAT5 upregulation. FIG. 2B is the result of Western blot analysis of the lysate of A549 cells 6 hours after transfection by treatment with X-NC and X-NP, and, shows that NFAT5 protein expression significantly increased compared to other control groups while β-actin expression did not change. When the NFAT5 protein band was compared with that of untreated control cells, a significant increase of about 65% was shown in X-NC, suggesting that osmotic pressure played an important role in the introduction of NFAT5 (*P<0.05, one-way ANOVA). FIG. 2C shows the comparison of osmotic pressure of X-NC, X-NP, and PEI. FIG. 2D is a still image of a real-time image of a cell, and each labeled plasma membrane, nucleus, and X-NC/DNA^{YOYO} transfected portion (indicated with an arrow) can be confirmed. This suggests that X-NC shows a pathway for cell internalization without disrupting the membrane or through endocytosis (X-NC bound to vesicles is not observed), and the magnification is 100×, and scale is 10 μm.

FIGS. 3A-3E show that dexamethasone (Dex) affects the transfection efficiency of X-NC by inhibiting NFAT5. In the process of NFAT5 inhibition in A549 cells using dexamethasone (hereafter Dex, 10⁻⁶ M), dexamethasone inhibits the transfection efficiency of osmotic molecules. FIG. 3A is the result of FACS analysis of cells transfected by treatment with nanocomplexes of X-NC/GFP, X-NP/GFP, and PEI25k/GFP. In the group treated with X-NC and X-NP, GFP expression was reduced in cells in which NFAT5 was suppressed by the osmotic activity of the treated materials, and cells transfected with PEI25k were not affected by the inhibitory materials. FIG. 3B is the result of representing the percentage of GFP-transfected cells after inhibition through Dex. GFP expression was reduced by 85% by X-NC, by 80% by X-NP, and not decreased by PEI25k at all. FIG. 3C is the result of Western blot analysis of NFAT5-inhibited cells after 48 hours. A decrease in GFP expression was found and data were expressed as the mean of three independent experiments plus the standard deviation (*P<0.05, ****P<0.0001, one-way ANOVA). FIG. 3D is an image taken 48 hours after transfection after NFAT5 inhibition treatment through Dex in A549 cells. In the case of the osmotic substances X-NC and X-NP, GFP expression was decreased, and in the case of PEI25k, it was confirmed that Dex treatment did not affect transfection (scale: 500 μm). FIG. 3E showed that NFAT5 expression was decreased in Dex-treated cells with osmotic X-NC and X-NP compared to cells not treated with Dex in immunofluorescence staining analysis at 24 hours after transfection. PEI25k shows no significant NFAT5 expression, which is due to the lack of osmotic activity, which is not different from that treated with Dex (Scale: 50 μm).

FIGS. 4A-4I show the transfer process of X-NC^{T}/tGFP through an in vitro BBB and BTB (BBB/BTB) microfluidic chip model, and the penetration of X-NC^{T}/tGFP through the BBB/BTB under the condition of a flow rate of 0.1 μl/min in the microfluidic chip model. FIG. 4A is a diagram of the BBB/BTB microfluidic chip model, and FIG. 4B is the composition of the BBB/BTB model. FIG. 4C is a confocal microscope image comparing the expression levels of X-NC^{T}/tGFP and GFP accumulated in the central part of the chip through the BBB, respectively at 120 minutes after starting to perfuse the treated material into the chip and at 48 hours after transfection. FIG. 4D is a confocal microscope image comparing the expression levels of X-NC^{T}/tGFP and GFP accumulated in the central part through the BTB, respectively at 120 minutes after starting to perfuse the treated material with or without NFAT5 inhibitor (Dex), and at 48 hours after transfection. FIG. 4E shows that the transmittance of X-NC^{T} is more improved than that of X-NP^{T} in the change of fluorescence intensity generated while the nanocomplex penetrates the BBB. FIG. 4F suggest that the fluorescence intensity change in the central part of the chip when X-NC^{T} penetrated the BTB significantly reduced the penetration ability of the material by the NFAT5 inhibitor. FIG. 4G showed that X-NC^{T} had a higher permeability compared to X-NP^{T} in the BBB permeability measurement data. FIG. 4H showed that the BTB permeability measurement data showed negligible insignificant permeability in the presence of an inhibitor. FIG. 4I represent GFP fluorescence intensity represented as % when the nanocomplex was permeabilized for 48 hours against BBB/BTB on a microfluidic chip model. (n=3, Error bars represent standard deviation) (**P<0.01; ***P<0.001; ****P<0.0001, one-way ANOVA)

FIGS. 5A-5B show the distribution process of X-NC in vivo. In vivo distribution of X-NC/pGL3 was confirmed by tracing luciferase protein expression after IP injection into 6-week-old nude Balb/c mice (n=4). FIG. 5A shows that X-NCs are particularly distributed in the brain in a biofluorescence image taken one week after drug treatment. FIG. 5B is data showing the distribution of X-NC in various organs through the expression level of luciferase protein, represented in RLU/mg units. This suggests that luciferase protein was significantly expressed by drug treatment in brain tissue (n=4, Error bars represent standard deviation) (*P<0.05; **P<0.01; ***P<0.001, one-way ANOVA).

FIGS. 6A-6C show the cell death initiation process after SHMT1 inhibition in glioblastoma (GBM) cells expressing luciferase. FIG. 6A is a biofluorescence image of GBM cells stably expressing luciferase transfected with X-NP/siSHMT1, X-NC/siSHMT1 and X-NP/siScr in a 6-well plate. After 48 and 72 hours, fluorescence was minimally expressed in the X-NC-treated experimental group, suggesting that SHMT1 enzyme inhibition was maximized and cell death occurred in this experimental group. FIG. 6B shows that, after SHMT1 inhibition, in the luciferase expression data quantified and measured in a chemiluminometer, cells transfected with X-NC show the least expression, and thus show the maximum SHMT1 inhibition effect (n=3, Error bars represent standard deviation) (***P<0.001, one-way ANOVA). In the results of TUNEL assay of FIG. 6C to compare the apoptosis effect, it can be confirmed that the most apoptosis-inducing effect occurred in the process of siSHMT1 delivery by X-NC, which can be confirmed through the brown-stained nucleus (magnification: 4×, 10×). Phase contrast images show that many cell deaths are the result of the inhibitory effect of SHMT1 enzyme by X-NC (magnification: 10×, scale: 100 μm).

FIGS. 7A-7D show an in vivo treatment method for inducing apoptosis of GBM by suppressing SHMT1 expression using X-NC in mouse brain (n=4). The timeline of 7A is a treatment guideline for transplanted brain tumors. It shows a series of processes from transplantation on day 1, starting drug treatment after the brain tumor has settled 2 weeks later, and confirming the effect of the treatment method at week 4. In the biofluorescence image analysis, it can be confirmed that brain tumor growth was maximally inhibited by the delivery of siSHMT1 (15 μg) through X-NC, as the fluorescence intensity rapidly decreased after 4 weeks. FIG. 7B shows that GBM transfected with X-NC/siSHMT1 showed reduced brain tumor volume and decreased fluorescence expression by 97%. It showed results compared to the X-NP/siSHMT1 group, which decreased by 62%, and the control group, in which fluorescence expression increased rapidly. FIG. 7C is the result of Western blot analysis of SHMT1 protein in brain tissue lysate treated with nanocomplex. It indicates that there is no change in β-actin protein expression and that SHMT1 protein expression is greatly reduced in mice treated with X-NC than in X-NP and control groups. When the SHMT1 protein band of cells treated with X-NC was compared to the band of untreated control cells by density analysis, X-NC showed a significant reduction of SHMT1 by 87%, indicating inhibition of brain tumor. Data are presented as the mean standard deviation of three independent experiments. (***P<0.001, one-way ANOVA). FIG. 7D is a comparison of tissue morphologies between normal tissues of mouse brain, heart, kidney, and liver and when nanochains loaded with siSHMT1 entered into each tissue.

FIG. 8 is a diagram showing the process of synthesizing polydixylitol polymer nucleic acid transporter (PdXYA), which is the first skeleton of the present invention.

FIG. 9 is a diagram showing the cytotoxicity evaluation results for X-NC. (A) in FIG. 9 is the result of evaluating cytotoxicity by comparing the cell viability of PEI25k/DNA, X-NC/DNA, and X-NP/DNA complexes according to the N/P ratio. (B) in FIG. 9 is the result of evaluating cytotoxicity by comparing the cell viability of X-NP/DNA (N/P 20) (left in the figure) and X-NC/DNA (N/P 20) (right in the figure) complexes in human umbilical vein endothelial cells (HUVEC), astrocytes, and glioblastoma (GBM).

FIG. 10 is a diagram showing electrophoretic migration analysis for the verification of RNase protection of X-NC.

FIG. 11 shows the composition of the in vitro BBB/BTB microfluidic chip system and the comparison result of the fluorescence intensity according to the movement of the material. (A) in FIG. 11 shows the fluorescence intensity in the vessel channel of X-NC^{T} in the BBB model of the chip. (B) in FIG. 11 shows the fluorescence intensity in the vessel channel of X-NP^{T} in the BBB model, (C) in FIG. 11 shows the fluorescence intensity in the vessel channel of X-NC^{T} in the BBB model when Dex is not treated, and (D) in FIG. 11 shows the fluorescence intensity in the vessel channel of X-NC^{T} in the BBB model when Dex is treated. (E) in FIG. 11 shows the morphology of the BBB model, and (F) in FIG. 11 shows the morphology of the BTB model.

FIG. 12 is a diagram showing the results of induction of GBM in which luciferase is stably expressed.

(A) in FIG. 12 indicates that luciferase is not expressed in GBM. (B) in FIG. 12 is a diagram showing the results of induction of GBM in which luciferase is stably expressed.

FIG. 13 is a diagram showing the process of transplanting a brain tumor into a 5-week-old nude male Balb/c mouse.

FIG. 14 is a diagram showing full-size bioluminescent images of mouse GBM brain tumors 4 weeks after treatment by treating X-NP/siSHMT1 and X-NC/siSHMT1 to kill cancer cells through SHMT1 inhibition.

### BEST MODE

Hereinafter, preferred embodiments of the present invention will be described with reference to the accompanying drawings. However, the embodiments of the present invention can be modified in many different forms, and the scope of the present invention is not limited to the embodiments described below.

Inspired by previous studies on polydixylitol-based vectors with high osmotic activity, the present inventors developed a nucleic acid transporter in a nanochain form (X-NC) that allows gene drugs to cross the BBB and enter tumors. The nucleic acid transporter in a nanochain form obtained by the present invention can enable the release of a gene drug without external support. In addition, there is an advantage in that a gene can be delivered to each cell by passing through the BBB and BTB, and improved gene transfection can be performed by loading a large amount of genes due to the delivery vehicle having an improved aspect ratio.

The term ‘nucleic acid transporter’ of the present invention may be used interchangeably with ‘gene transporter’.

The nucleic acid transporter in a nanochain form of the present invention is a nucleic acid transporter in a nanochain form (X-NC) to which polydixylitol polymer (PdXYP) of Chemical Formula I is linearly linked.

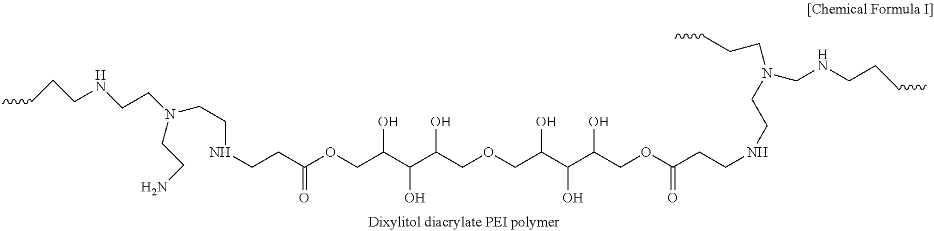

The high aspect ratio of the nucleic acid transporter in a nanochain form (X-NC) synthesized from polydixylitol nanoparticles (X-NP) with xylitol dimers as an octamer analogue increases an effective nucleic acid loading capacity with the cumulative effect of osmotic pressure. In addition, high osmotic properties of the flexible and linear X-NC can enhance the passage efficiency for BBB and BTB and improve cell entry ability.

In addition, activation of nuclear factor of activated T cells-5 (NFAT5), which is involved in protecting cells from osmotic stress triggered by the accumulation of osmolytes (e.g.: polyols), can be applied as an additional advantage of X-NC. NFAT5 activates carriers and channels to restore the osmotic equilibrium of the membrane, thereby promoting translocation of the BBB and BTB of X-NCs and cellular uptake.

In the present invention, the polydixylitol polymer nucleic acid transporter (PdXYP), which has been previously developed, is improved and manufactured in a chain form to be designed to deliver genes.

The nanochain may be in the form of a nanochain represented by Chemical Formula II below. In this case, n may be an integer of 2 to 100, for example, 2 to 10, preferably 3 to 5.

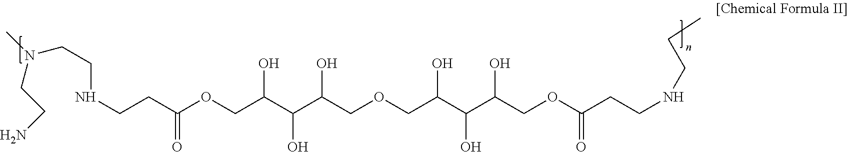

For example, the nucleic acid transporter of the present invention may have the following Chemical Formula III structure.

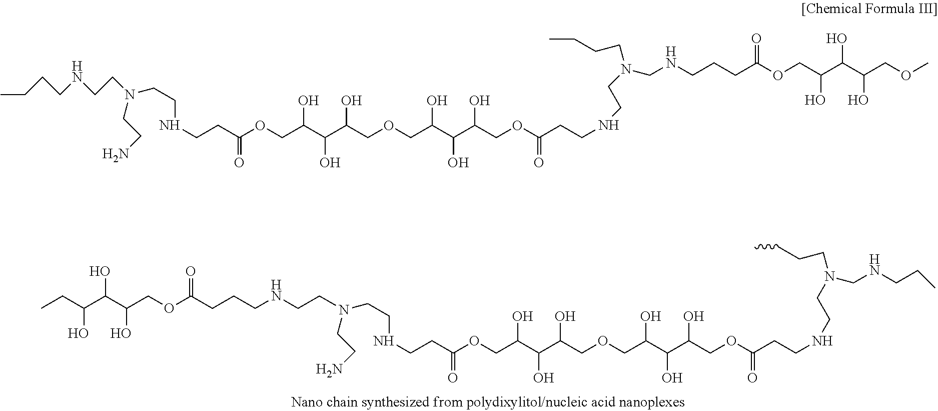

Such chain structure can be obtained through a step of mixing polydixylitol polymer (PdXYP) and dixylitol diacrylate (dXYdA). For example, it can be obtained by standing at 40 to 80° C., for example, at 60° C. for 6 to 48 hours after mixing polydixylitol polymer (PdXYP) and a cross-linking agent at a molar ratio of 1:4 to 6, preferably 1:5.

Furthermore, it may further comprise a step of mixing the nucleic acid transporter in a nanochain form (X-NC) with the therapeutic nucleic acid, wherein the therapeutic nucleic acid and the nucleic acid transporter in a nanochain form (X-NC) are mixed at a molar ratio of 1:0.5 to 1:100.

At this time, dixylitol diacrylate (dXYdA) has a structure of the following Chemical Formula IV. When this linkage is used, X-NC in which the PdXYP nucleic acid transporter is connected in a chain form by Michael addition reaction is prepared.

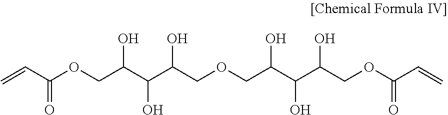 [SMILES: C=CC(=O)OCC(O)C(O)C(O)COCC(O)C(O)C(O)COC(=O)C=C]

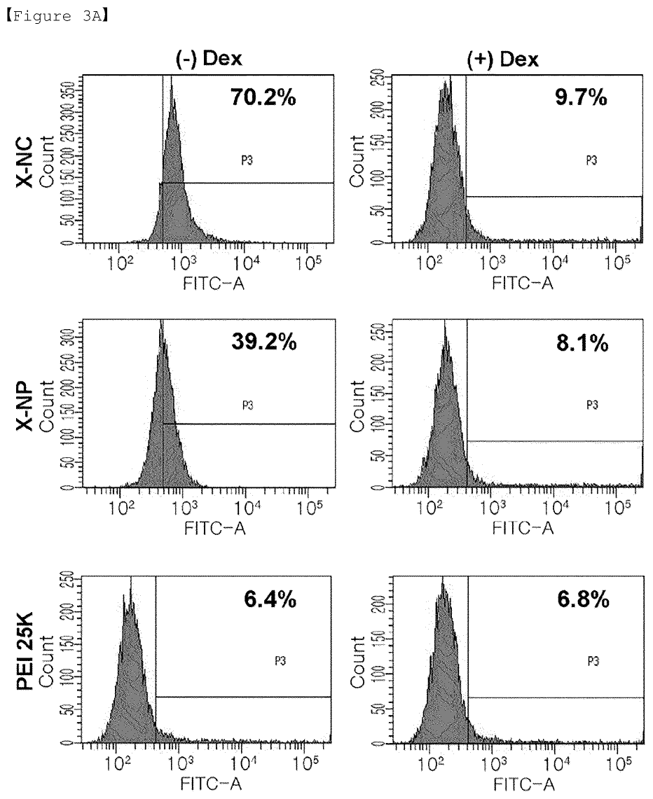

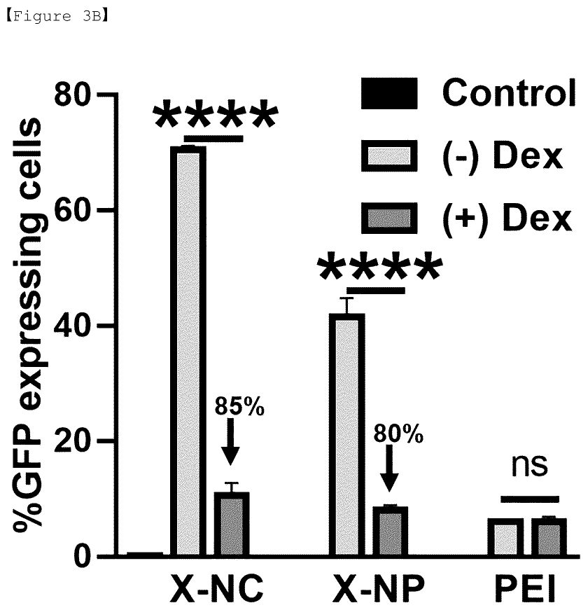

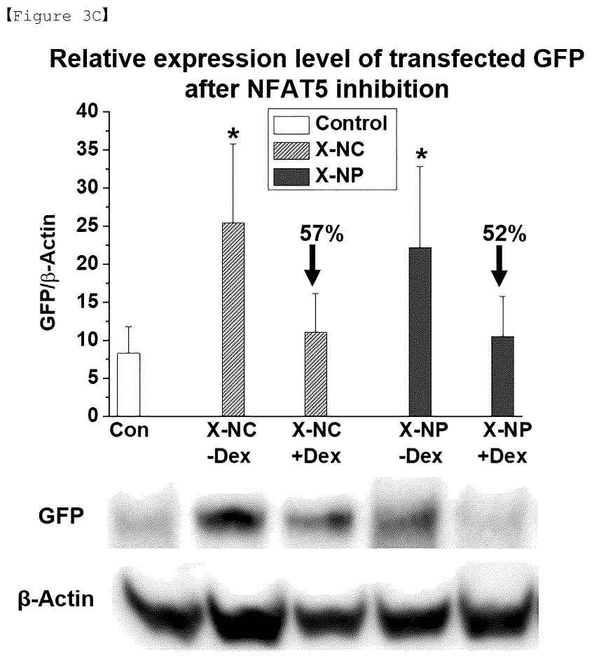

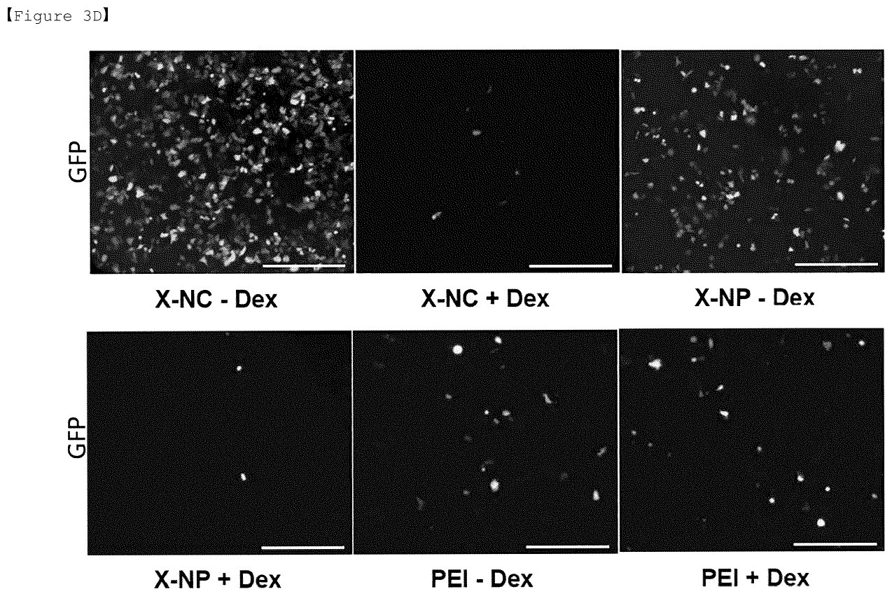

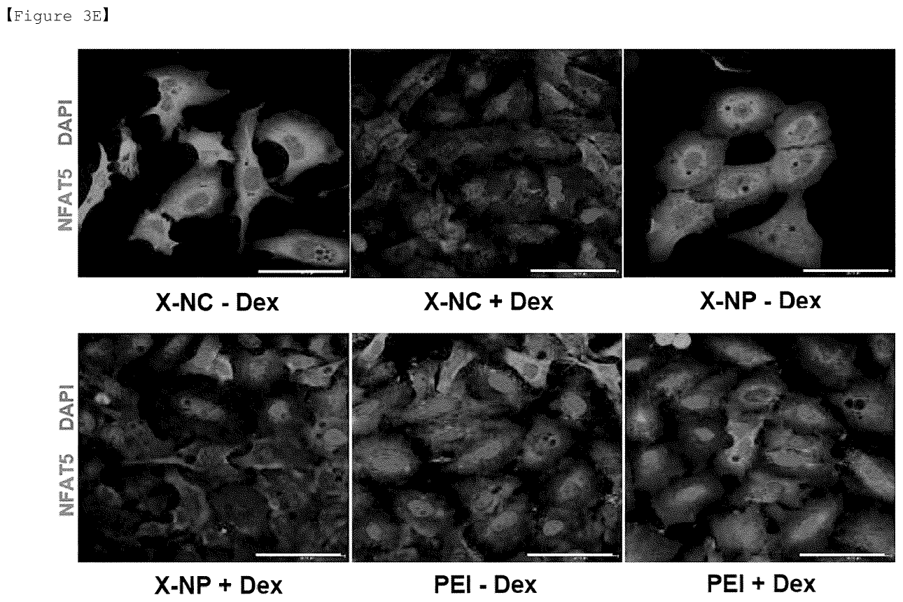

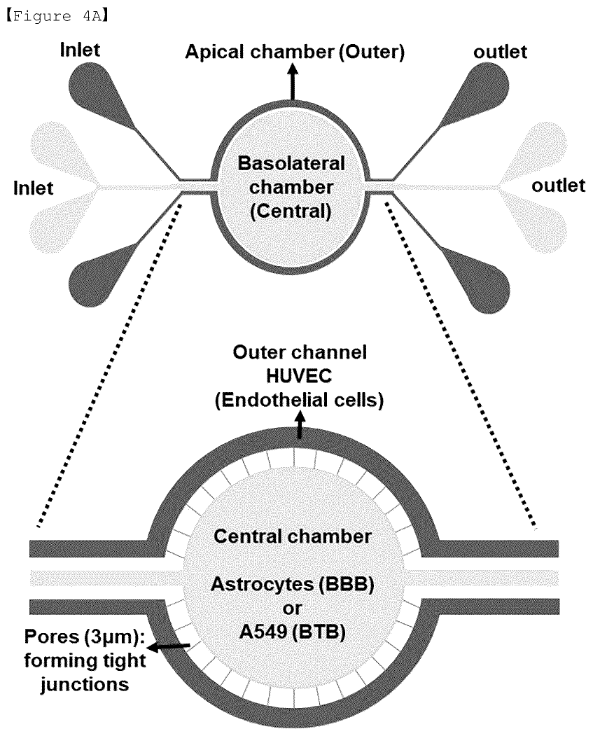

In the present invention, the term “polydixylitol polymer nucleic acid transporter (polydixylitol polymer based nucleic acid transporter, PdXYP) is a gene transporter patented by the present inventors (Korean Patent No. 10-1809795). This transporter can be prepared by preparing di-xylitol through acetone/xylitol condensation method, preparing dixylitol diacrylate (dXYA) through esterification of the di-xylitol with acryloyl chloride, and then reacting the dixylitol diacrylate and low molecular polyethylenimine (PEI, 1.2 kD) through Micheal addition reaction. In addition, by additional Micheal addition reaction between dXYP and PdXYP, nano molecules can be prepared in a nanochain form. (FIG. 3)

The term, “xylitol” refers to a kind of sugar alcohol-based natural sweetener having a chemical formula of C₅H₁₂O₅. It is extracted from birch and oak trees and has a unique pentose structure. To prepare the polydixylitol polymer nucleic acid transporter of the present invention, di-xylitol, an xylitol dimer, was used.

The term “acryloyl chloride” may also be referred to as 2-propenoyl chloride or acrylic acid chloride. The compound reacts with water to produce acrylic acid, reacts with sodium carboxylate to form anhydride, or reacts with alcohol to form an ester group. In a specific embodiment of the present invention, Dixylitol diacrylate (dXYA) was formed by reacting acryloyl chloride with di-xylitol, a dimer of xylitol, a type of sugar alcohol.

The term “polyethylenimine (PEI)” is a cationic polymer having primary, secondary and tertiary amino groups and having a molar mass of 1,000 to 100,000 g/mol. It effectively compresses anionic nucleic acid to form colloidal particles, and has high gene delivery efficiency due to its pH-responsive buffering ability, so that genes can be effectively delivered to various cells in vitro and in vivo. In the present invention, polyethylenimine may be linear-type represented by Chemical Formula V or branched-type represented by Chemical Formula VI below, and its molecular weight is low molecular weight, preferably 50 to 10,000 Da (based on weight average molecular weight) in consideration of cytotoxicity. Polyethylenimine is soluble in water, alcohol, glycol, dimethylformamide, tetrahydrofuran, esters, etc., and insoluble in high molecular weight hydrocarbons, oleic acid, and diethyl ether.

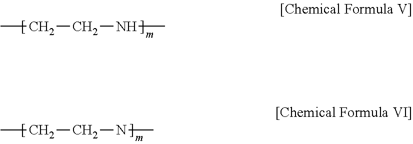

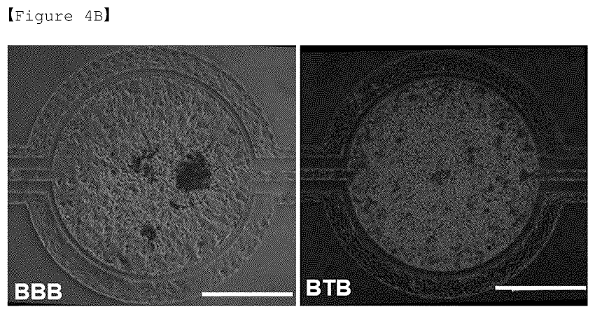

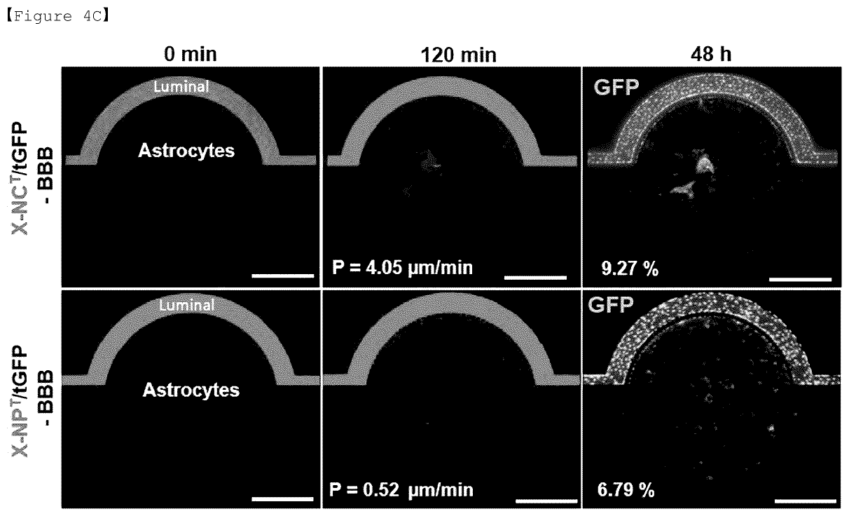

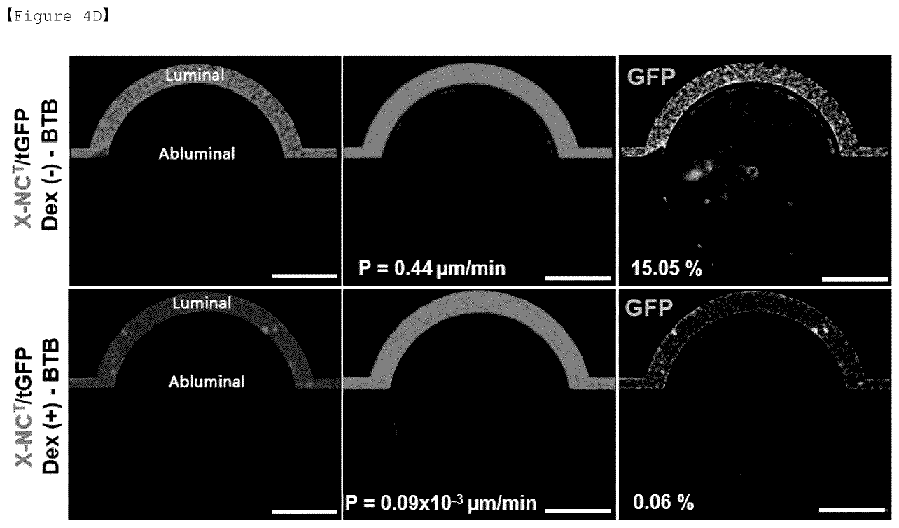

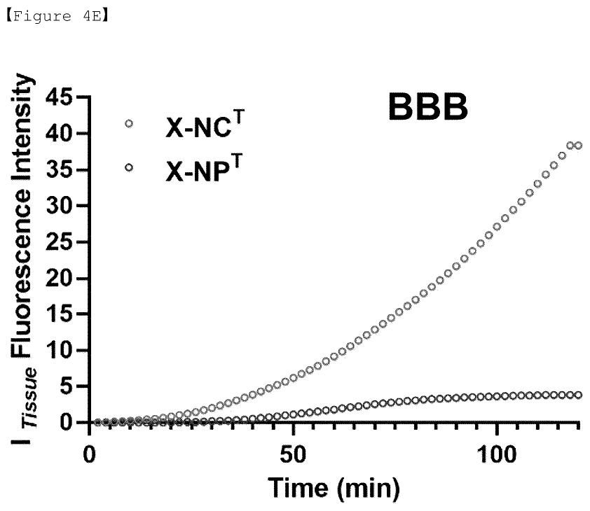

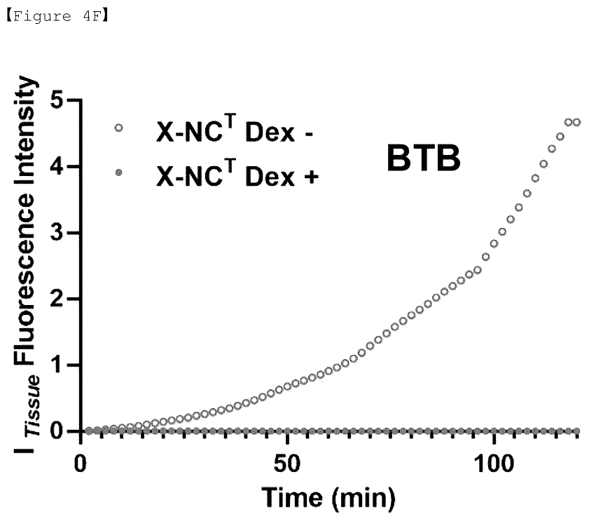

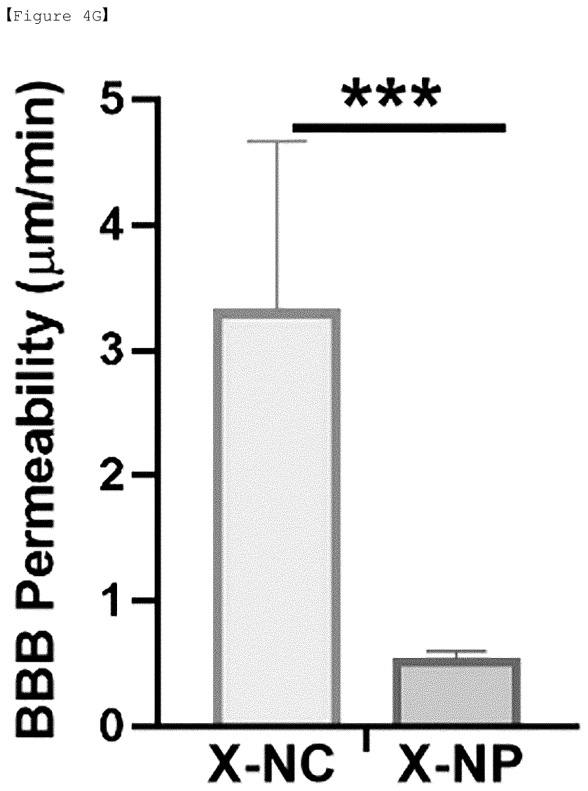

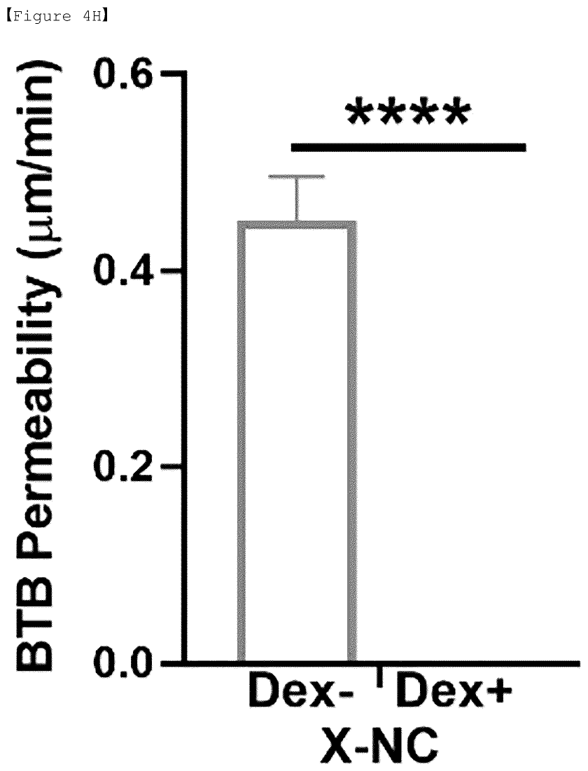

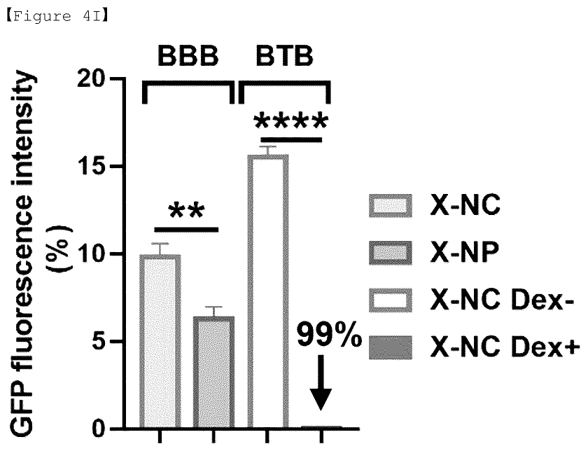

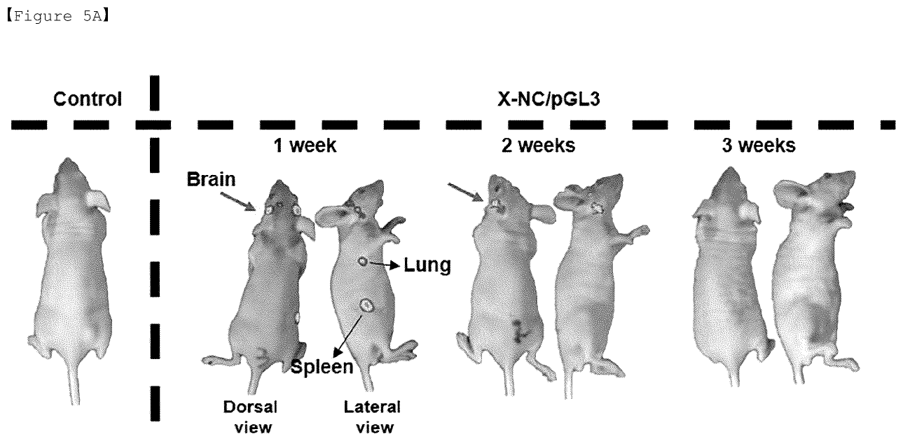

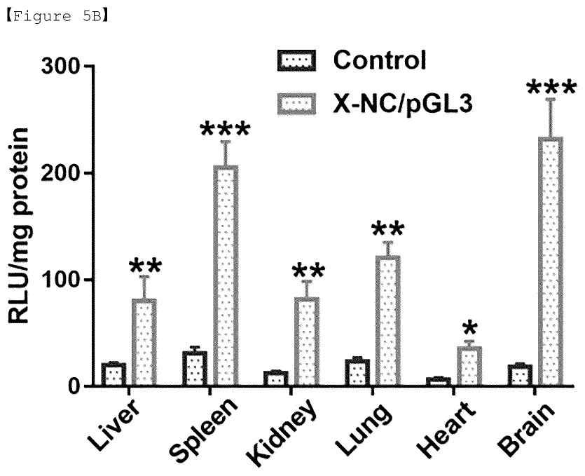

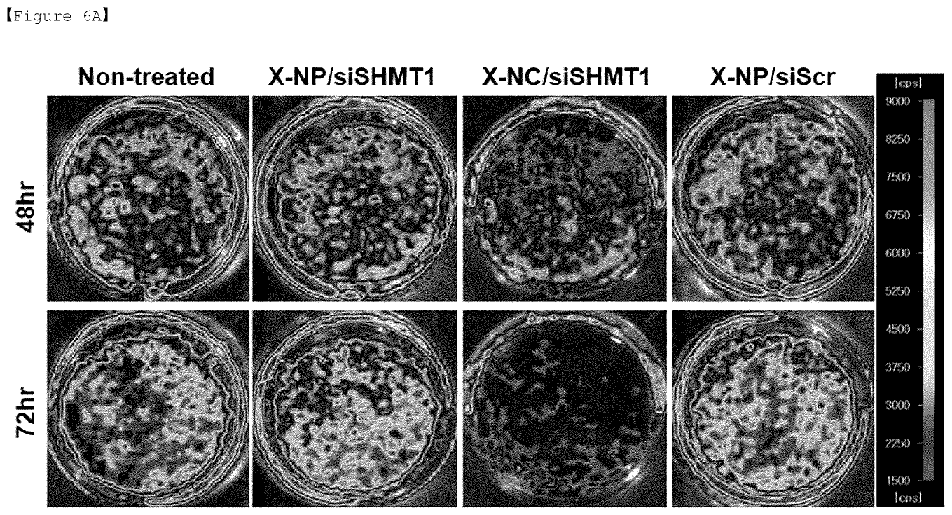

Compared to X-NP nanoparticles, it has been confirmed that the polymer X-NC nanochain of the present invention with a high aspect ratio has improved characteristics such as more effective gene loading and high permeability, and has enhanced gene delivery ability. The nucleic acid transporter in a nanochain form of the present invention is a non-spherical particle that causes rotational motion as well as tumbling and rotation resulting in translational motion, preventing motion and adhesion to cells and providing high transformation potential. In addition, the linear and flexible shape of X-NC has the advantage of extended systemic circulation and thus easily avoids phagocytosis by macrophages. This provides sufficient time for X-NC to pass through the BBB and BTB (FIG. 5 and FIG. 4C).

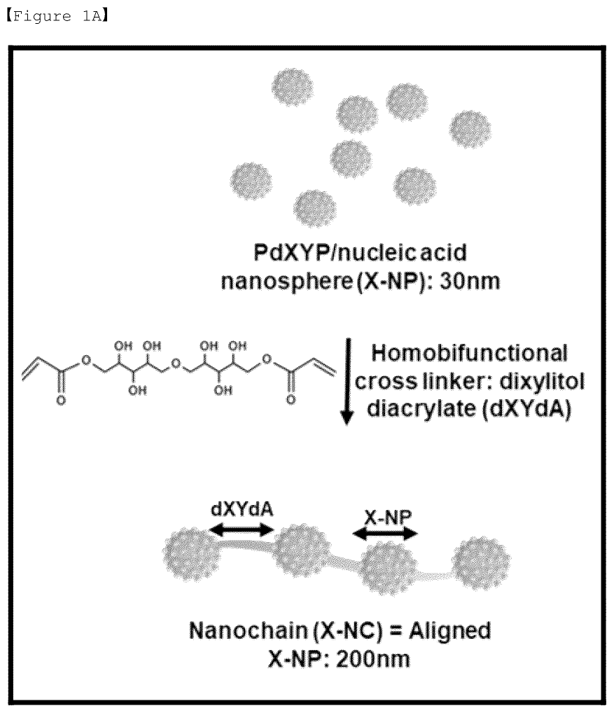

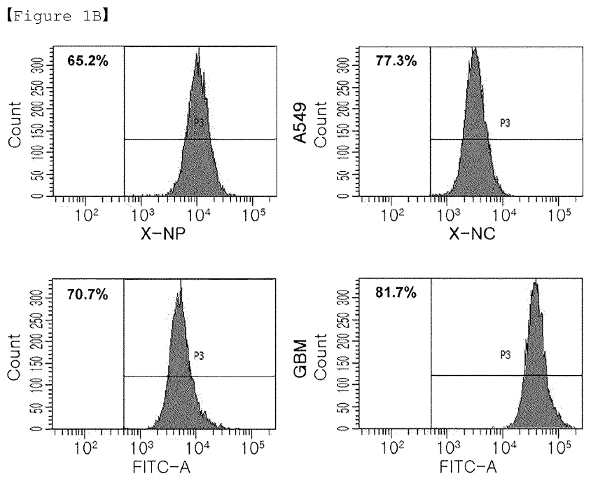

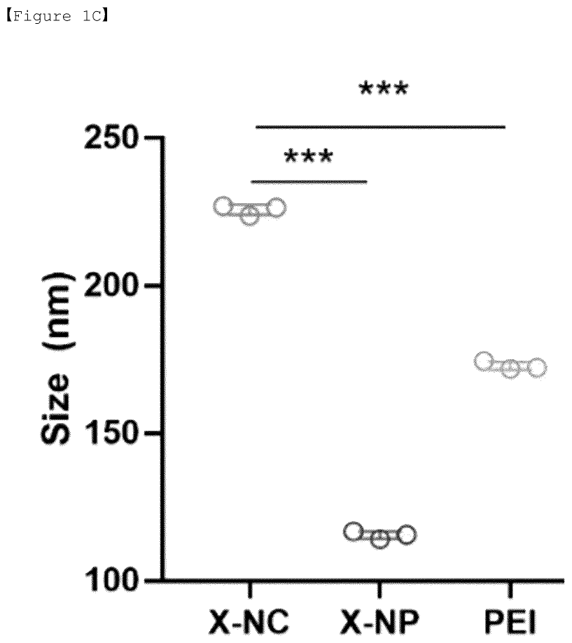

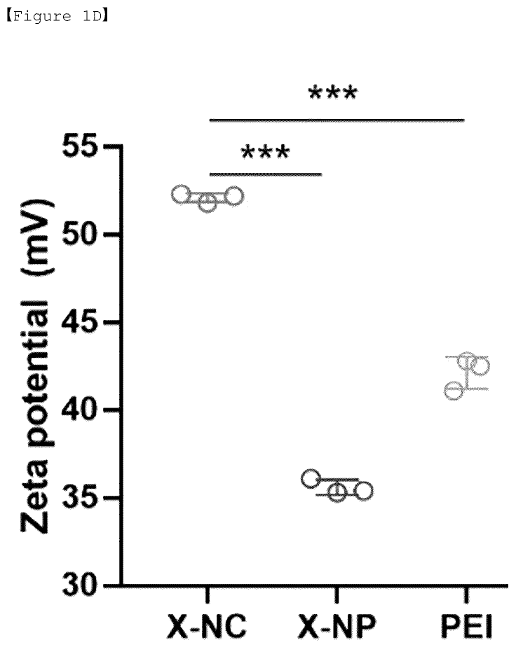

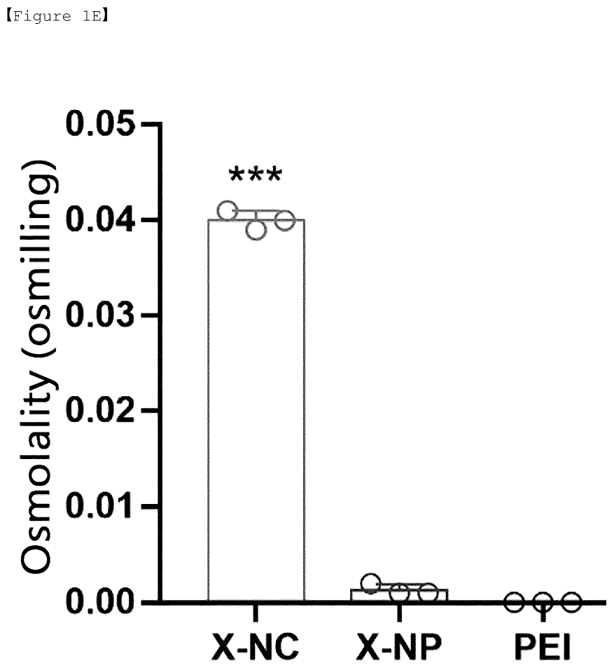

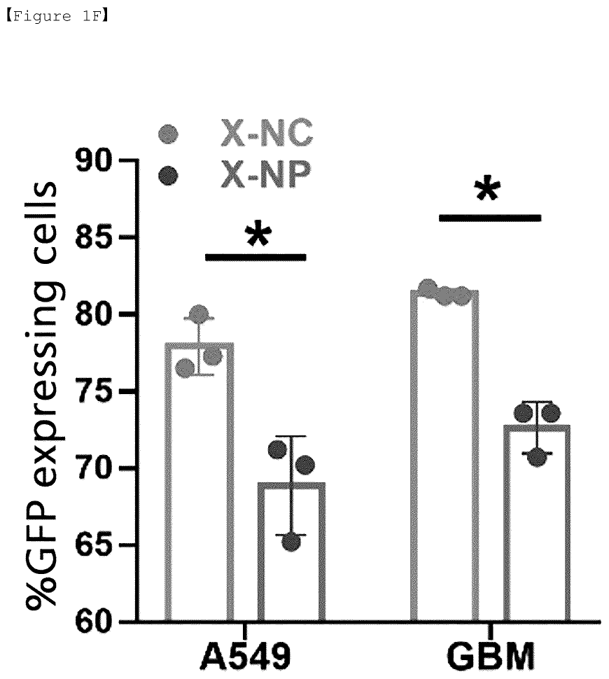

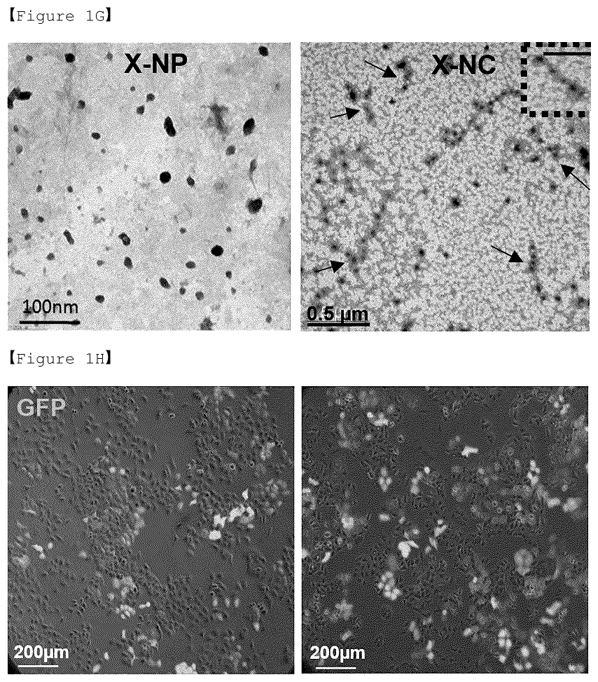

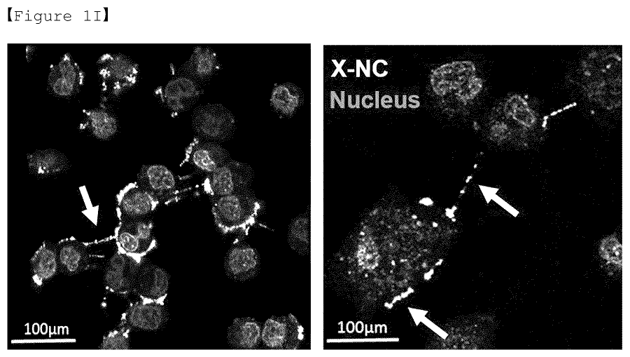

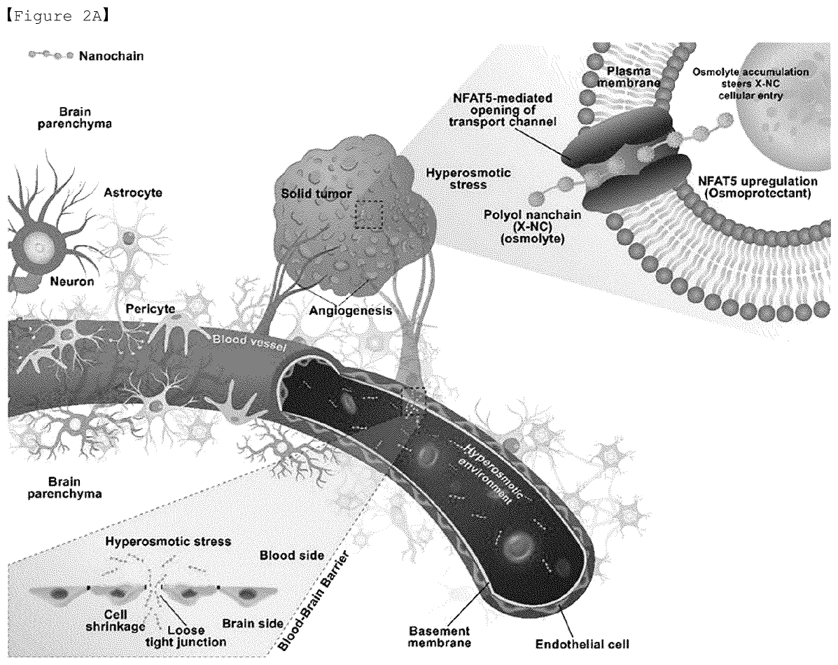

The nucleic acid transporter in a nanochain form X-NC of the present invention (˜200 nm) exhibits aggregated nanoparticles (˜30 nm), but X-NC exhibits enhanced transfection (FIG. 1B, FIG. 1H). In addition, it easily passes through the BBB and BTB (FIG. 4C, FIG. 4D), suggesting that a spatially aligned nanochain is better than a simple spherical aggregate of nanoparticles.

Thus, the ordered geometry of X-NC combined with the focused hyperosmotic effect increases its ability to migrate across the BBB and/or BTB and penetrate into cells. X-NC induces the activation of channels used to enter cells. X-NC exhibits an average of 2-fold higher intracellular hyperosmotic effect than other NPs, which activates osmotic protective signaling pathways to prevent cell contraction and damage by generating hyperosmotic stress that disrupts homeostasis in the vicinity of cells.

An important role in the osmotic protection of cells is played by activation of NFAT5, which initiates the intracellular transport of osmolyte molecules such as polyols across the cell membrane. NFAT5 promotes transport of organic osmolytes that can be utilized by X-NC in the uptake process by activating carriers and/or channels to restore membrane equilibrium. As can be seen in the Examples, cells transfected with X-NC show up-regulation of NFAT5 by 65% after 6 hours. Therefore, the gene transporter of the present invention is a nanochain composed of a plurality of nanoparticles with high osmotic properties, which improves the movement and transfection ability to the BBB and/or BTB by an NFAT5-mediated mechanism.

As another aspect, the gene transporter of the present invention may be in the form of a nanocomplex forming a complex with a therapeutic nucleic acid.

Furthermore, the present invention provides a pharmaceutical composition for gene therapy comprising the nucleic acid delivery nanocomplex in which the therapeutic nucleic acid is coupled to the X-NC as an active ingredient. The pharmaceutical composition of the present invention can be used for treatment or prevention of diseases for which gene therapy is possible depending on the type of therapeutic nucleic acid constituting the pharmaceutical composition.

For example, the therapeutic nucleic acid may be at least one selected from the group consisting of siRNA (small interfering RNA), shRNA (small hairpin RNA), esiRNA (endoribonuclease-prepared siRNAs), anti-sense oligonucleotide, DNA, single-stranded RNA, double-stranded RNA, DNA-RNA hybrid and ribozyme. For example, the therapeutic gene may be SHMT1 siRNA.

For example, X-NC loaded with hydroxymethyltransferase small interfering RNA (Serine hydroxymethyltransferase, SHMT1 siRNA) can show remarkable therapeutic results in the treatment of brain tumor mouse models by silencing SHMT1 function and inducing tumor cells to apoptosis. The X-NC of the present invention having high aspect ratio can overcome the limitations of BBB and BTB penetration and tumor penetration and can be a promising approach for desired therapeutic outcomes.

The high aspect ratio of the gene transporter of the present invention increases the loading capacity of an effective gene drug and can spontaneously form a nanocomplex with nucleic acid. The nucleic acid transporter of the present invention not only enables an increase in the loaded amount of the gene to be delivered, but also promotes the passage of the BBB and the function of absorption into the cell by using the high osmotic property.

According to another aspect of the present invention, a pharmaceutical composition for gene therapy containing a nucleic acid transporter as an active ingredient is provided. For example, the pharmaceutical composition for gene therapy is for cancer therapy.

The pharmaceutical composition of the present invention can be administered together with a pharmaceutically acceptable carrier, and may additionally include a binder, a lubricant, a disintegrant, an excipient, a solubilizer, a dispersant, a stabilizer, a suspending agent, a coloring agent, a flavoring agent, and the like in addition to the above active ingredients when administered orally. In the case of an injection, the pharmaceutical composition of the present invention may be used by mixing a buffer, a preservative, a soothing agent, a solubilizer, an isotonic agent, a stabilizer, and the like. In addition, when administered topically, the composition of the present invention may use a base, an excipient, a lubricant, a preservative, and the like.

The formulation of the composition of the present invention may be prepared in various ways by mixing with a pharmaceutically acceptable carrier as described above, and in particular, it may be prepared for inhalation administration or injection administration. For example, for oral administration, it can be prepared in the form of tablets, troches, capsules, elixirs, suspensions, syrups, wafers, etc., and in the case of injections, it can be prepared in unit dosage ampoules or multiple dosage forms. It can be formulated into other solutions, suspensions, tablets, pills, capsules, sustained-release preparations, and the like. Drug delivery through inhalation is one of the non-invasive methods, and therapeutic nucleic acid delivery through an inhalation administration formulation (e.g., aerosol) is advantageously used for the treatment of a wide range of lung diseases, in particular. This is because the anatomy and location of the lungs allow immediate, non-invasive access and local application of the gene delivery system without affecting other organs.

On the other hand, examples of carriers, excipients and diluents suitable for formulation may include lactose, dextrose, sucrose, sorbitol, mannitol, xylitol, erythritol, malditol, starch, acacia, alginate, gelatin, calcium phosphate, calcium silicate, cellulose, methyl cellulose, microcrystalline cellulose, polyvinylpyrrolidone, Water, methylhydroxybenzoate, propylhydroxybenzoate, talc, magnesium stearate or mineral oil, etc.

The pharmaceutical composition of the present invention can be administered orally or parenterally. The administration route of the pharmaceutical composition according to the present invention is not limited to these, but for example, oral, intravenous, intramuscular, intraarterial, intramedullary, intrathecal, intracardiac, transdermal, subcutaneous, intraperitoneal, intestinal, sublingual or topical administration is possible. For such clinical administration, the pharmaceutical composition of the present invention can be formulated into a suitable formulation using known techniques. For example, for oral administration, it may be administered by mixing with an inert diluent or an edible carrier, sealing in a hard or soft gelatin capsule, or pressing into a tablet. For oral administration, the active ingredient may be mixed with an excipient and used in the form of ingestible tablets, buccal tablets, troches, capsules, elixirs, suspensions, syrups, wafers and the like. In addition, various formulations for injection, parenteral administration, etc. can be prepared according to known techniques or commonly used techniques in the art.

The effective dosage of the pharmaceutical composition of the present invention varies in its range depending on the patient's weight, age, sex, health condition, diet, administration time, administration method, excretion rate, and severity of the disease, and can be easily administered by a person skilled in the art.

For example, the pharmaceutical composition of the present invention may be in the form of a nanocomplex in which the therapeutic nucleic acid is loaded on the nucleic acid transporter in a nanochain form of the present invention to form a complex with the therapeutic nucleic acid, wherein the therapeutic nucleic acid is SHMT1 siRNA (esiRNA, Cat No: 111430)

The pharmaceutical composition of the present invention may have a therapeutic or preventive effect on cancer stem cells depending on the type of therapeutic nucleic acid constituting the invention, and the cancer may be selected from the group consisting of lung cancer, bone cancer, pancreatic cancer, skin cancer, head and neck cancer, skin melanoma, uterine cancer, ovarian cancer, rectal cancer, colorectal cancer, colon cancer, breast cancer, uterine sarcoma, fallopian tube carcinoma, endometrium carcinoma, cervix carcinoma, vagina carcinoma, vulva carcinoma, esophageal cancer, small intestine cancer, thyroid cancer, parathyroid cancer, soft tissue sarcoma, urethral cancer, penile cancer, prostate cancer, chronic or acute leukemia, pediatric solid tumor, differentiated lymphoma, bladder cancer, kidney cancer, renal cell carcinoma, renal pelvic carcinoma, primary central nervous system lymphoma, myelencephalon tumor, brain stem glioma, and pituitary gland adenoma.

As another aspect, the present invention provides a genetic cancer cell treatment method using the polydixylitol polymer nucleic acid transporter in a nanochain form of the present invention described above, a nucleic acid transporter complex comprising the same, or a pharmaceutical composition comprising the same.

### MODE FOR CARRYING OUT THE INVENTION

Hereinafter, the present invention will be described in more detail through Examples. These Examples are only for illustrating the present invention, and the scope of the present invention is not construed as being limited by these examples.

1. Reagents and Materials Used

In this experiment, the following materials and reagents were used to prepare a polydixylitol-based polymer nanochain nucleic acid transporter (Nanochain synthesized from polydixylitol polymer based nucleic acid transporter, hereinafter referred to interchangeably with ‘X-NC’ and ‘nanochain’) in which the polydixylitol polymer nucleic acid transporters of the present invention (polydixylitol polymer based nucleic acid transporters, hereinafter referred to interchangeably with ‘PdXYP’, ‘X-NP’ and ‘polydixylitol polymer’) is linked in a chain form, and to confirm its effect.

The reagents used in this experiment such as bPEI (branched Poly(ester imine), Mn: 1.2k and 25k), DMSO (dimethyl sulfoxide), acryloyl chloride, xylitol, 4′-deoxypyridoxine hydrochloride, sodium cyanoborohydride (NaCNBH4), genistein, chlorpromazine bafilomycin A1 and MTT (3-(4,5-dimethyl thioazol-2-yl)-2,5-diphenyl tetra-zolium bromide) were Sigma (St.Louis, MO, USA) products. In addition, a luciferase reporter encoding firefly (Photonus pyralis) luciferase, pGL3-vector and enhancer were purchased from Promega (Madison, WI, USA). The green fluorescent protein (GFP) gene was purchased from Clontech (Clontech, Palo Alto, CA, USA). For confocal microscopic analysis, TRITC (Tetramethylrhodamine isothiocyanate) and YOYO-1 iodide (Molecular Probes, Invitrogen, Oregon, USA) dyes were used. Scramble siRNA (siScr) was purchased from Genolution Pharmaceuticals Inc. (Republic of Korea), and SHMT1 siRNA (siSHMT1) was purchased from Thermo Fisher Scientific (USA).

2. Synthesis of Polymer Nanochain

The polydixylitol polymer nanochain nucleic acid transporter (X-NC) according to the present invention was synthesized through the following steps. The nucleic acid transporter of the present invention was invented by improving and enhancing the patented material previously invented by the inventors. Therefore, the registered patent (Korean Patent No. 10-1809795) can be cited until the following steps 2-3.

2-1. Synthesis of Di-Xylitol

The present inventors focused on the fact that the number and stereochemistry of hydroxy groups affect cell-to-cell delivery, and attempted to develop a gene delivery material with increased intracellular delivery efficiency by controlling osmotically active hydroxy groups. As there is no commercially available sugar alcohol having 8 hydroxyl groups, the present inventors directly synthesized an xylitol dimer, di-xylitol, as an octamer analogue through the process of FIG. 1.

Specifically, xylitol was first crystallized into diacetone xylitol (Xy-Ac) crystals using the acetone/xylitol condensation method of Raymond and Hudson. The terminal hydroxy group of diacetone xylitol was reacted with trifluoromethyl sulphonyl chloride (CF₃SO₂—O—SO₂CF₃) to produce trifluoromethane sulphonyl xylitol (TMSDX). The prepared trifluoromethane sulphonyl xylitol was reacted with diacetone xylitol in an equal molar amount in the presence of dry tetrahydrofuran (THF) to form di-xylitol diacetone (Xy-Ac dimer). This reaction product was finally converted to xylitol dimer by opening the ring of the compound in HCl/MeOH solution (FIG. 1(a)).

2-2. Synthesis of Dixylitol Diacrylate

Dixylitol diacrylate (dXYA) monomer was synthesized by esterifying di-xylitol with 2 equivalents of acryloyl chloride. Di-xylitol (1 g) was dissolved in dimethylformamide (DMF) (20 mk) and pyridine (10 ml), and an acryloyl chloride solution (dissolved 1.2 ml in 5 ml DMF) was added dropwise at 4° C. with constant stirring to prepare an emulsion. After the reaction was completed, the HCl-pyridine salt was filtered off, and the filtrate was added dropwise to diethyl ether. The product precipitated as a syrup and dried under vacuum.

2-3. Synthesis of Polyxylitol Polymer (PdXYP)

The polyxylitol polymer (PdXYP) of the present invention was prepared through a Michael addition reaction between low molecular weight bPEI (Poly ethylene imide, 1.2k) and dixylitol diacrylate (dXYA).

Specifically, synthesized dXYA (0.38 g) dissolved in DMSO (5 ml) was added dropwise to 1 equivalent of bPEI (1.2 kDa, dissolved in 10 ml DMSO) and reacted at 60° C. with constant stirring for 24 hours. After the reaction was completed, the mixture was dialyzed against distilled water at 4° C. for 36 hours using a Spectra/Por membrane (MWCO: 3500 Da; Spectrum Medical Industries, Inc., Los Angeles, CA, USA). Finally, the synthesized polymer was lyophilized and stored at −70° C.

2-4. Synthesis of Nanochain (X-NC)

In order to cross-link the polydixylitol polymer (PdXYP) nanoparticles (X-NP) obtained in 2-3 above into X-NC nanochains, dixylitol diacrylate (dXYdA) was used as a cross-linking agent. More specifically, dXYdA cross-linking agent was added to the X-NP solution at a molar ratio of polydixylitol polymer (PdXYP):cross-linking agent (dXYdA) of 1:5, and then left at 60° C. overnight. The molar concentrations of the cross-linking agent and PdXYP were tightly controlled to maintain the linear alignment of the self-assembled X-NC nanochains. Later, the nanochain was dialyzed for 24 hours using a 3.5 kDa dialysis membrane to exclude unreacted cross-linking agent. The resulting polydisperse mixture suspension of nanochains (X-NC) was centrifuged (10,000 g) to precipitate large particles, and nanochains were obtained in the supernatant.

More specifically, as shown in FIG. 8, the first step of the three-step synthesis of polydixylitol-nanochain (X-NC) is a step of synthesis of polydixylitol-PEI (PdXYP) by combining di-xylitolduacrylate (dXYdA) and bPEI (1.2 kDa). Furthermore, as shown in FIG. 1A, the PdXYP (X-NP) may be cross-linked using a cross-linking agent (dXYdA) at a molar ratio of 1:5. A nucleic acid-loaded polydixylitol nanochain (X-NC) can be formed using a molar ratio of X-NP:dXYdA of 1:5. Furthermore, by centrifuging the mixed suspension containing X-NC, nanochains of uniform size can be obtained from the supernatant.

The nanochain synthesis method as described above was proposed considering the design criterion of high aspect ratio at the nanometer scale (≤200 nm).

3. Characterization Analysis of Nanochain

(1) TEM Image

Through the TEM image, it can be confirmed that the nanoparticles (X-NP) obtained in 2 above are circular nanoparticles with a size of about 30 to 50 nm (FIG. 1G, left), and the nanochains (X-NC) are linear and have a size of about 150 to 200 nm (Arrow) (FIG. 1G, right). This suggests that the size of the X-NC is more than 3 X-NPs in length, and that the X-NC is composed of more than 3 X-NPs connected to each other, as shown in the inset image (FIG. 1G, top right).

It was verified that the physical size of the X-NC measured by DLS and the X-NP constituting thereof was the same as the TEM result (FIG. 1C).

(2) Toxicity

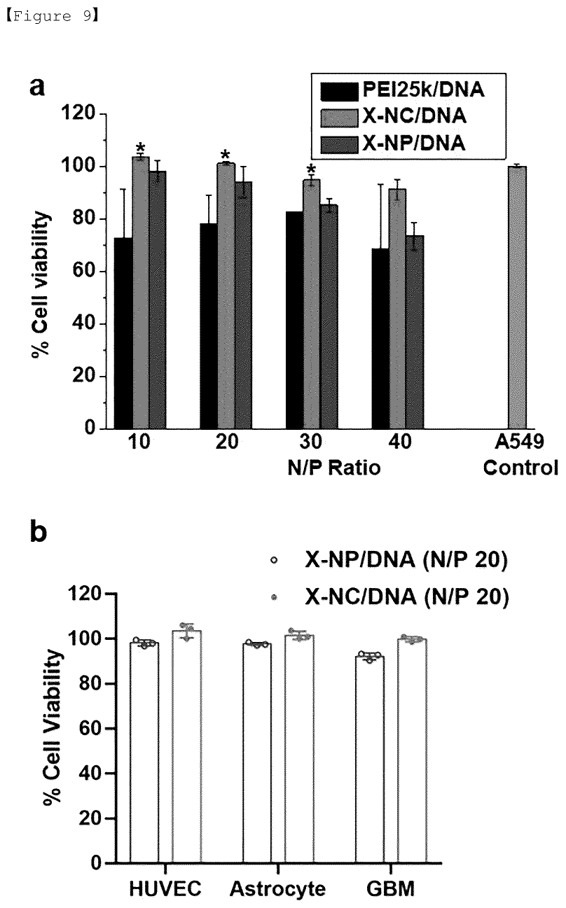

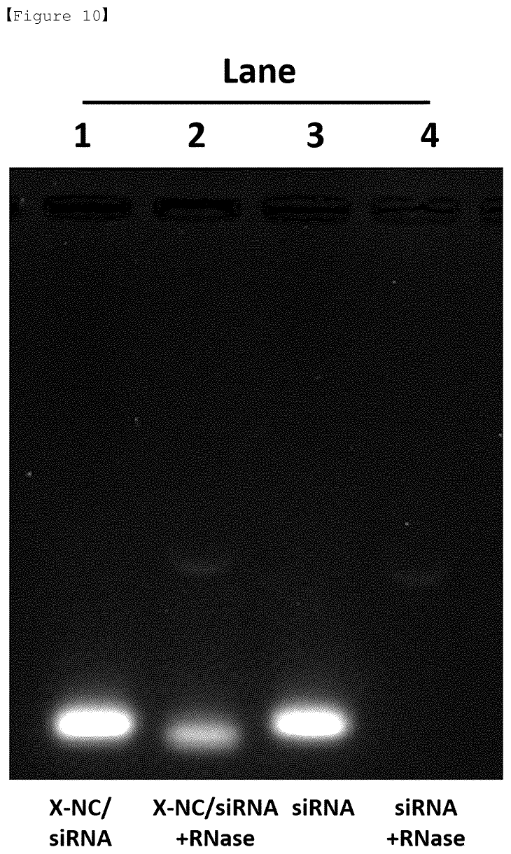

It was confirmed that the nanochain (X-NC) showed a high surface charge density of 52 mV compared to the nanoparticle (X-NP) (35 mV) or PEI (polyethylenimine) (40 mV) (FIG. 1D), but there was no toxic effect on cells (FIG. 9).

This is presumably because the charge density of the X-NP constituting the X-NC is lower than that of the non-bonded X-NP, so it has a minimal detrimental effect on the cell membrane. In addition, the hydroxyl group forms intramolecular hydrogen bonds that can protect X-NC from high surface charge, further enhancing cell viability.

This experiment was performed on A549 cancer cells, and untreated A549 cells were used as a control. In FIG. 9, the N/P ratio refers to the ratio of transporter and nucleic acid. On the other hand, PEI25k/DNA means DNA delivery using a PEI25k transporter, X-NC/DNA means a complex in which a nanochain transporter delivers DNA, and X-NP/DNA means a nanoparticle transporter delivers DNA. In this case, pGL3 was used as the DNA.

On the other hand, FIG. 9B is an evaluation of cytotoxicity by analyzing the cell viability of X-NP/DNA (N/P 20) (left in the figure) and X-NC/DNA (N/P 20) (right in the figure) complexes. Here, HUVEC refers to human umbilical vein endothelial cells, and Astrocyte refers to astrocyte, and GBM refers to glioblastoma.

FIG. 10 is the result of electrophoretic migration analysis for the verification of RNase protection of X-NC. Lane 1 represents X-NC/siRNA, Lane 2 represents X-NC/siRNA+RNase-treated group, which means that the nanochain siRNA is protected from the nucleic acid degrading enzyme RNase. Lane 3 represents pure siRNA that is not loaded on the nanochain, and Lane 4 means that when RNase is added to this siRNA, the siRNA is not protected and is degraded by nucleic acid degrading enzyme. As a result, it can be seen that high surface charge can be strongly coupled electrostatically to protect nucleic acid from degradation by nucleic acid degrading enzyme (FIG. 10).

(3) Osmotic Pressure

The osmotic pressure of the X-NC, X-NP (N/P 20) and PEI25k (N/P 10) nanocomplexes was measured in both water and cell culture media at various times after transfection using a cryoscopic osmometer 030 (Gonotec, USA). Measurements were performed at 0 min, 5 min, 15 min, 30 min, 1 hr, 5 hr, 7 hr, 9 hr, 24 hr and 30 hr after transfection, and the measurement result was calculated in mOsm through the descent of the freezing point.

As can be seen in FIG. 1E, the nanochain (X-NC) was found to have 40 times higher osmotic pressure than the nanoparticle (X-NP) or PEI complex in distilled water, suggesting the increased high osmotic pressure property of X-NC.

(4) Other Characteristics

Since the nanochains (X-NC) (˜80%) have a chain-like/linearly ordered shape with high aspect ratio, hyperosmotic pressure, optimal size (≤200 nm) and high surface charge, it showed a higher transfection rate compared to individual X-NPs (˜65%). FIG. 1B indicates that X-NC shows higher transfection efficiency than X-NP in the FACS data of A549 and GBM cells treated with nanochain. FIG. 1F is the percent efficiency data of the GFP transfection efficiency measured by FACS, and FIG. 1H shows transgenic GFP expression in human lung cancer cells (A549) (Scale: 200 μm) (FIG. 1B, F, H).

In addition, 60 minutes after transfection of the nanochain into the BBB/BTB microfluidic chip in vitro, the process of uptake of substances into cells was confirmed by perinuclear accumulation test (brightly lit, indicated by arrows) (FIG. 4C, 1I).

4. Entry of Highly Osmotic Nanochains (X-NC) into Cells

After transforming the nanochain into A549 cells, the osmotic pressure of the A549 cell medium was checked at various time points.

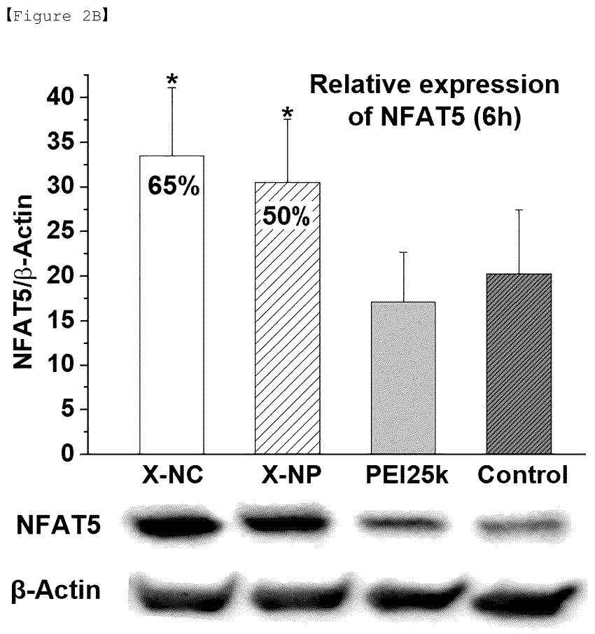

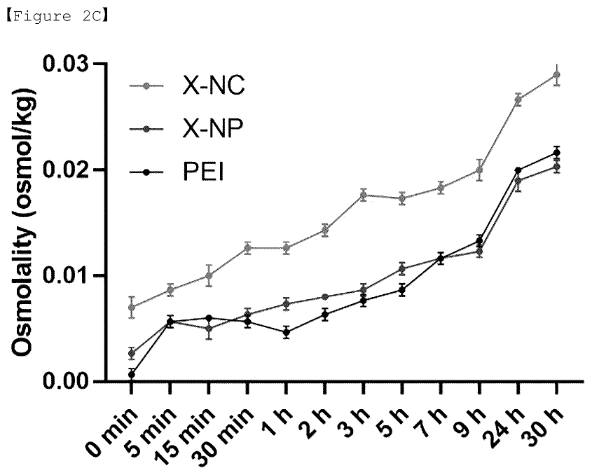

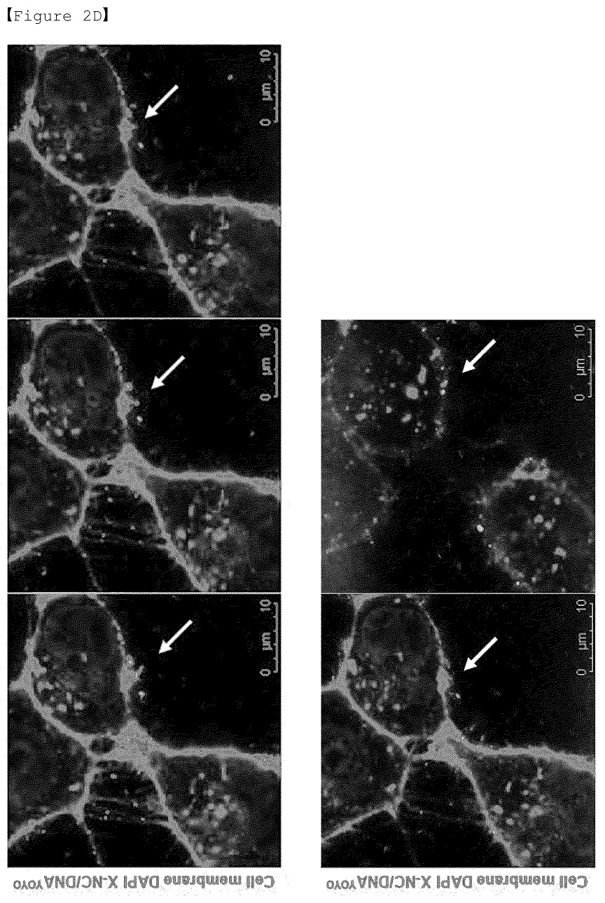

At any given time point, the permeability of X-NC was up to 2-fold higher than that of the X-NP and PEI composites (FIG. 2C).

The high osmotic properties of X-NC induces cell entry as seen in the A549 cell image of FIG. 2D, showing that X-NCs labeled with YOYO dye (indicated by arrows) are struggling to enter the cells. X-NCs do not compromise the integrity of the cell membrane until it penetrates inside, implying that a new material transport channel is involved in the process of entry into the cell membrane.

According to the continuous observation, NFAT5 was upregulated by 65% and 50% (relative to control) in both A549 cells transfected with X-NC and X-NP after 6 hr, respectively, but there was no overexpression of NFAT5 in PEI-transfected cells (FIG. 2B).

This phenomenon suggests that NFAT5 is activated in response to hyperosmotic pressure, leading to the movement of X-NCs across the cell membrane and through an unknown channel (FIG. 2A).

5. Effect of NFAT5 Inhibition by Dexamethasone (Dex) on Hyperosmotic Gene Delivery

NFAT5 is the dominant transcription factor activated in response to cellular hyperosmotic stress, which transports polyol molecules (osmolytes) across membranes to restore homeostasis.

As quantified by FACS, GFP transfection was induced by treating A549 cells with X-NC/GFP, X-NP/GFP and PEI25k/GFP complexes, respectively, in the absence and presence of dexamethasone (Dex), an NFAT5 inhibitor. X-NC/GFP refers to a complex in which GFP is mixed with the gene transporter of the X-NC nanochain, X-NP/GFP refers to a complex in which GFP is mixed with the gene transporter of nanoparticles, and the PEI25k/GFP complex refers to a complex in which GFP is mixed with the PEI25k gene transporter. In this case, ‘PEI25k’ is PEI having a molecular weight of 25 kD.

As a result, in the presence of Dex, X-NC significantly reduced GFP transfection (85% reduction), and the X-NP complex reduced GFP transfection by 80%. However, PEI25k-mediated GFP delivery remained unaffected by inhibitors. FIG. 3A is the result of FACS analysis of cells transfected by treatment with nanocomplexes of X-NC/GFP, X-NP/GFP, and PEI25k/GFP. In the group treated with X-NC and X-NP, GFP expression was reduced in cells in which NFAT5 was suppressed by the osmotic activity of the treated materials, and cells transfected with PEI25k were not affected by the inhibitory materials. FIG. 3B shows the percentage of GFP-transfected cells as a percentage after inhibition through Dex. (−)Dex indicates the absence of Dex, and (+)Dex indicates the presence of Dex (FIG. 3A, B).

Post-transfection images also showed reduced GFP expression (bright areas) in each group transfected with X-NC and X-NP due to the inhibition of NFAT5, which increases the uptake of the hyperosmotic complex (i.e., a complex in which GFP is mixed with a gene delivery system of nanoparticles or nanochains), in contrast to the PEI25k-treated group (FIG. 3D).

Similar to this result, the GFP protein expression levels in the NFAT5 inhibition group of the X-NC/GFP and X-NP/GFP transfected cells were reduced by 57% and 52%, respectively (FIG. 3C). This suggests that NFAT5 is involved in the material uptake process.

Due to cytotoxicity, PEI25k-treated cells were mostly dead and could not extract sufficient protein, so they were excluded from the analysis.

Immunocytochemical analysis also showed reduced NFAT5 expression in Dex-treated cells of X-NC and X-NP compared to PEI25k 24 hr after transfection, a pattern consistent with the GFP transfection images (FIG. 3E). These results show that the transfection ratio reduced by NFAT5 inhibition in the X-NC-treated group is significantly higher than that in the X-NP-treated group, which suggests that NFAT5 is involved in regulating the cellular environment and eventually leads to X-NC uptake in response to osmotic transport.

6. Verification of Passing Ability of X-NC Using BBB and BTB Microfluidic Chip Models

The real-time migration potential of X-NC was determined using microfluidic BBB and BTB models that allow flow and induce shear stress in the outer vascular chamber and the astrocyte barrier (BBB) and interendothelial barrier (BTB) present in A549 cancer cells.

The in vivo microenvironment in the central tissue compartment (brain side) was reproduced with BBB and BTB models. FIG. 4A shows diagrams of the BBB and BTB microfluidic chip models, and FIG. 4B shows the configuration of the BBB and BTB models, and is an enlarged image with a scale of 500 μm (FIG. 4A, B). The porous structure between the two compartments of the microfluidic chip facilitates the exchange of biochemical substances to form a tight junction structure.

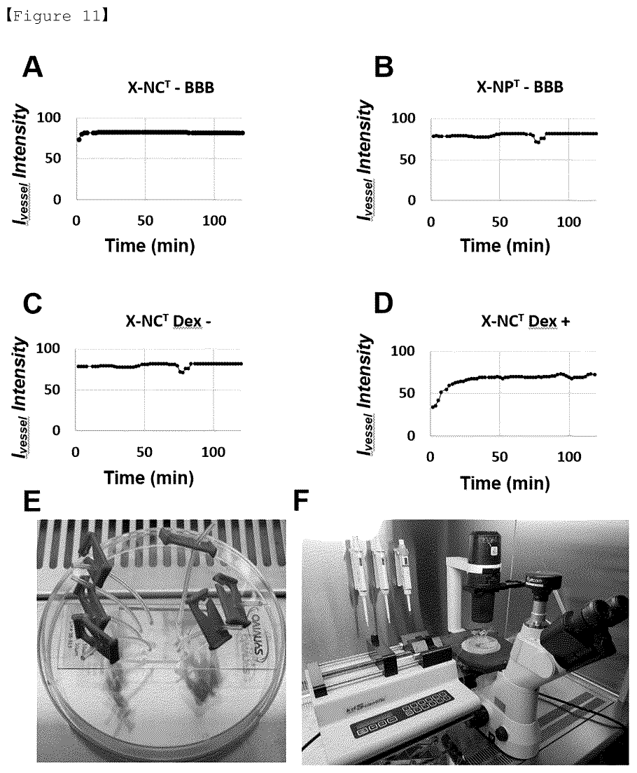

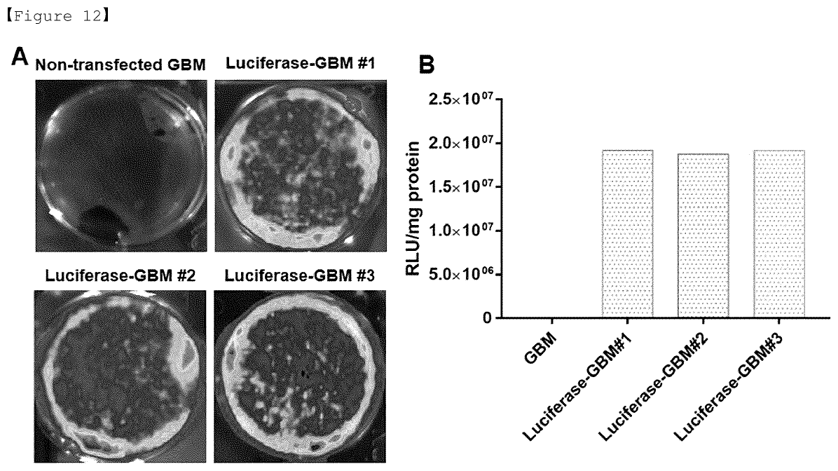

Meanwhile, FIG. 11A to FIG. 11D represent the fluorescence intensity in the vascular channel (vessel) of X-NC^{T} and X-NP^{T} in the BBB model of the microfluidic chip, respectively, and the fluorescence intensity decreased when Dex was treated. FIG. 11E is the morphology of the BBB model, and FIG. 11F shows the shape of the BTB model.

TRITC labeled vectors, X-NC^{T}/tGFP and X-NP^{T}/tGFP, were perfused through the vascular channels of the BBB model at a physiological flow rate of 0.1 μl/min, respectively. In this case, X-NP^{T} means that X-NC is tagged with a TRITC label. Linear accumulation of vector from 0 min to 120 min in the central compartment (I tissue) (brain side) in the BBB model shows higher fluorescence intensity in the X-NC^{T} perfusion chip than in the X-NP^{T} perfusion chip (FIG. 4E). In other words, it suggests that X-NCs show higher metastatic potential than X-NPs (FIG. 4C). This result shows that X-NC does not hinder the barrier passage compared to X-NP in the BBB, but rather greatly increases the barrier passage. The permeation of X-NCs through the vascular channel into the tissue chamber in the blood-to-brain direction was calculated as the transmittance (μl/min) using the following Equation (1):

P=(1−HC_{T})1/I_{V0}. V/S. dIt/dt Equation (1)
H_{CT}: Hematocrit number in the vascular channel (set to 0 because the medium does not contain blood cells)
I_{V0}: Fluorescence intensity of the outer vascular channel (apical channel)
Iₜ: Fluorescence intensity in the central compartment (basal side chamber) at a given time
v/S: Ratio of apex volume to surface area (0.1 cm)
dIt/dt: Changes in basal side intensity over time

The transmittance calculated by Equation (1) shows that X-NC has a higher transmittance (4.0544±μm/min) than X-NPs (0.516±μm/min) according to the fluorescence intensity accumulation data (FIG. 4G). In subsequent experiments, the efficiency of transfection of X-NCs across the BBB in the vascular channel into astrocytes in the tissue compartment (brain side) was assessed by the observed GFP expression. After 48 h, GFP expression of 9.3% observed in brain astrocytes indicates that X-NCs retain their function even after migration into the brain and have a higher transfection ratio than X-NPs (6.8%) (FIG. 4I).

The effect of the NFAT5 inhibitor Dex on the permeability of X-NCT across the BTB was also confirmed in the BTB microfluidic model. In the presence of Dex inhibitors, a rapid decrease in the accumulation of brain matter was observed for 120 minutes (FIG. 4D, FIG. 4F). In the BTB, the X-NCT transmittance was high when Dex was not treated, and the transmittance was greatly reduced when Dex was treated (FIG. 4H). No transfection was observed in chips later treated with Dex inhibitor (99% reduction, FIG. 4I), suggesting that NFAT5 plays an important role in migration and cellular uptake of X-NCs.

Another important observation is that the permeability of X-NCT through the BTB is lower than that through the BBB. This shows that a drug candidate is much more difficult to move to the BTB due to active efflux of the molecule.

7. Confirmation of the In Vivo Distribution of X-NC

In 6-week-old mice, X-NCs were loaded with pGL3 and injected intraperitoneally. One week after injection, the biodistribution profile determined by ex vivo tissue analysis showed distinct X-NC/pGL3 induced luciferase expression in the brain as well as in the spleen and lungs (FIG. 5B). In addition, through in vivo bioimaging (FIG. 5A), it can be seen that X-NC, which exhibits hyperosmotic characteristics by the di-xylitol group, crosses the BBB and expresses luciferase in the brain (arrow).

8. Tumor Growth Retardation Due to X-NC-Mediated SHMT1 Inhibition In Vitro and in a Brain Tumor Mouse Model

SHMT1, which is involved in DNA biosynthesis in tumor cells, is a surprising anticancer target that initiates apoptosis to prevent cell cycle and tumor mass proliferation. SHMT1 siRNA was loaded into X-NC, and a nanochain loaded with a composite therapeutic gene candidate (siSHMT1) was developed to inhibit the growth and proliferation of glioblastoma in brain tumor.

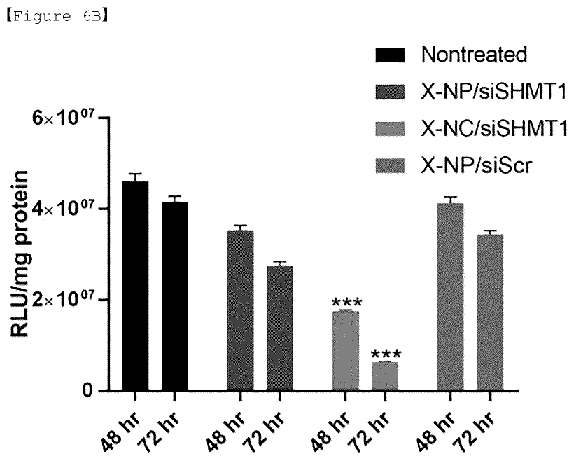

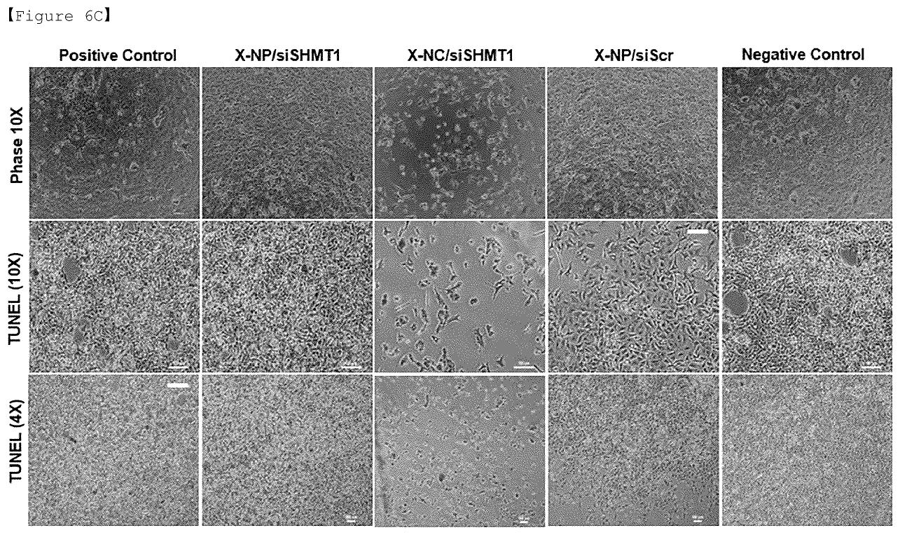

When GBM cells stably expressing luciferase (FIG. 12) were treated using SHMT1 siRNA-loaded X-NC (in vitro), better inhibition of SHMT1 (luciferase expression was shown to be the lowest) compared to the effect of X-NP (FIG. 6A). It was confirmed that siSHMT1 delivery was increased by X-NC, which effectively had higher gene loading capacity and improved transfection ability, leading to apoptosis of almost all cells 48 hours after transfection (FIG. 6C). Thus, X-NC treated cells showed a further decrease in luciferase expression after 72 hours due to sustained cell death in contrast to the X-NP treated group, and after silencing, untransfected cells resumed division after 48 hr. The scrambled siRNA delivery control showed no signs of a decrease in luciferase expression, but rather increased bioluminescence after 72 hours, suggesting consistent cell proliferation. The IVIS imaging results were validated by quantitative measurements obtained from protein extracts from the experimental group (FIG. 6B). Meanwhile, FIG. 12A indicates that luciferase is not expressed in GBM, and FIG. 12B indicates GBM in which luciferase is stably expressed.

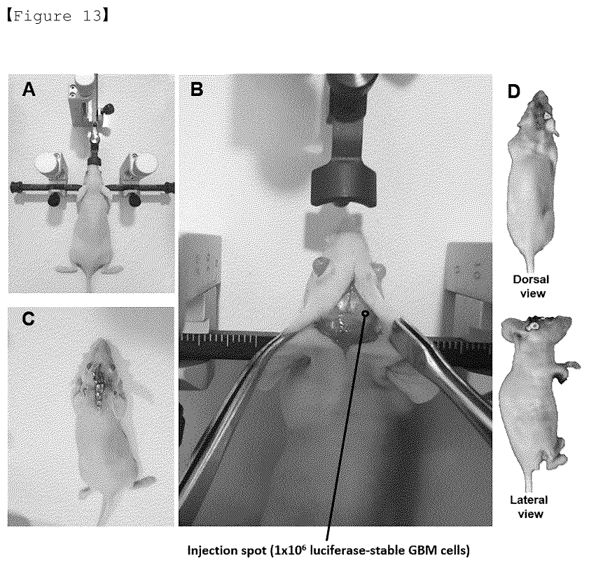

Luciferase-expressing brain tumor mice were treated with intraperitoneal administration of X-NC/siSHMT1 and X-NP/siSHMT1 2 weeks after tumor transplantation and bioluminescence images were observed weekly. The tumor transplantation process in brain tumor mice is shown in FIG. 13A, FIG. 13B and FIG. 13C, and FIG. 13D shows a luminescence image.

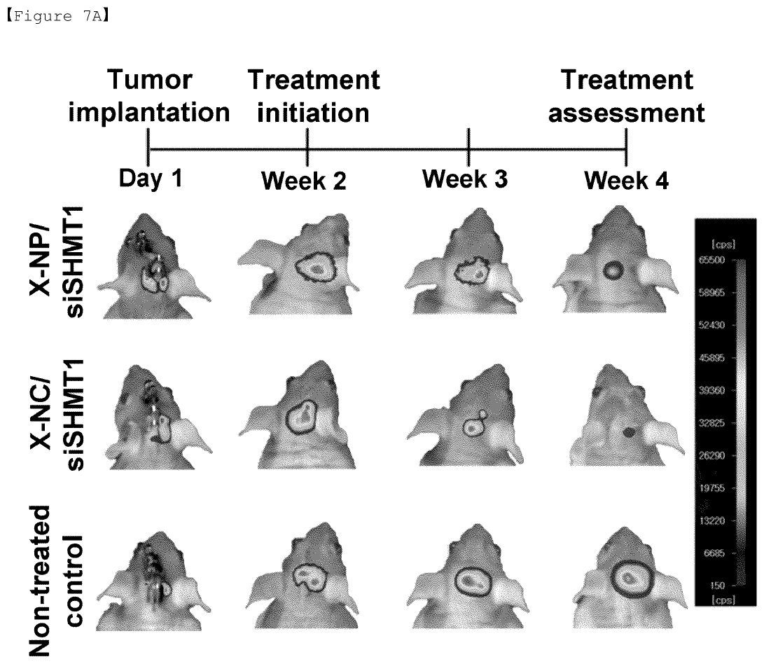

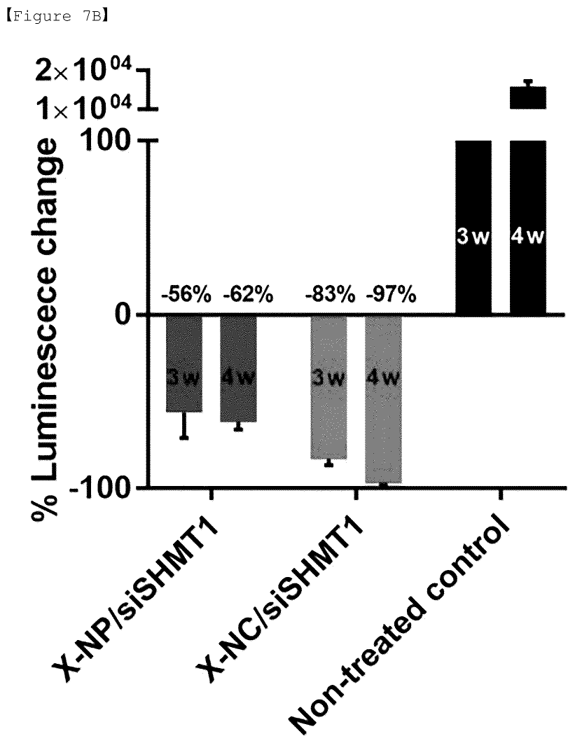

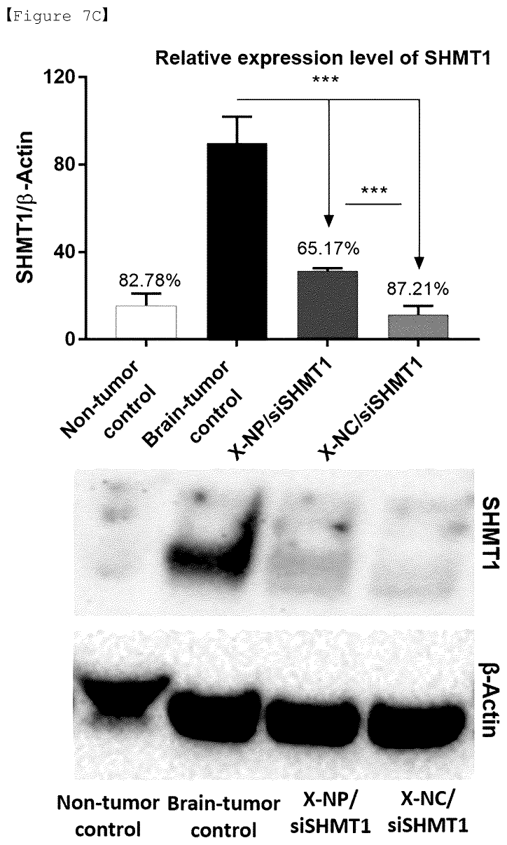

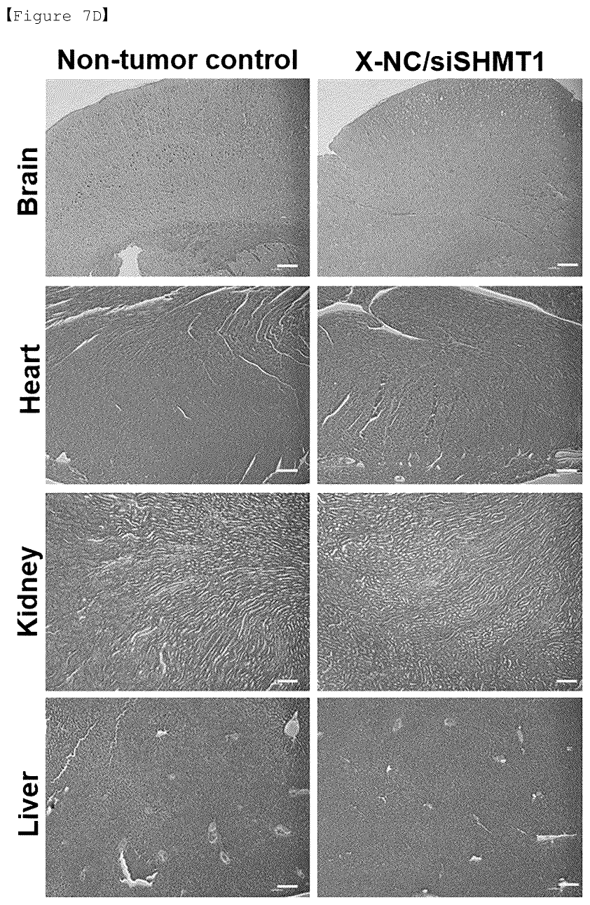

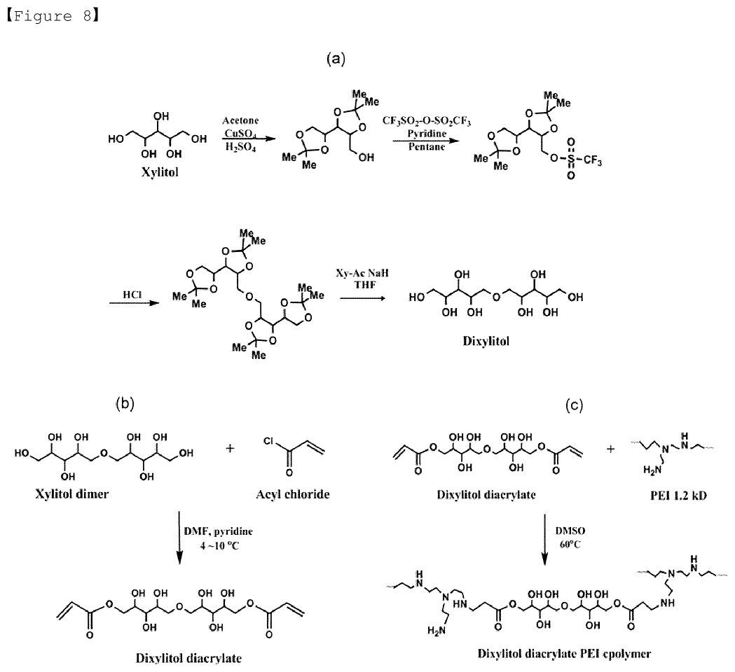

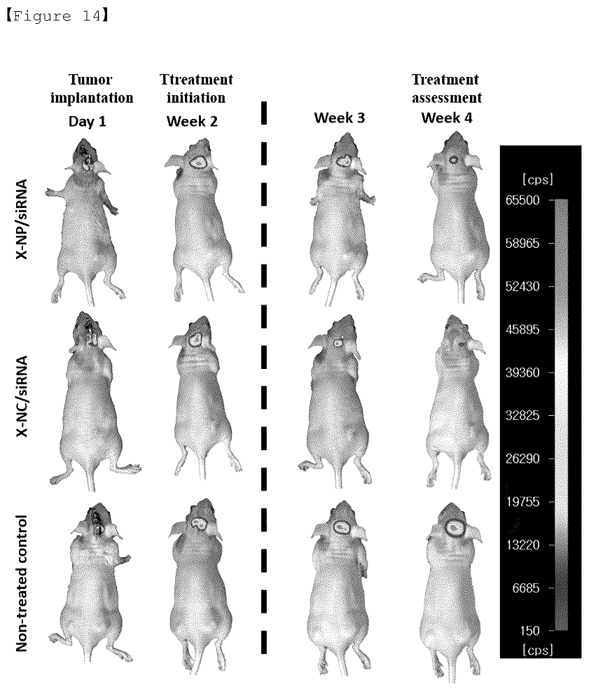

After 4 weeks of transplantation, bioluminescence intensity representing tumor volume significantly suppressed tumors by reducing tumor volume compared to the initial (Day 1) (compared to the initial luminescence on Day1 of the tumor) by 97% in X-NC-treated mice, compared to tumor volume reduction rate (62%) compared to the initial (Day 1) in X-NP-treated mice. In contrast, the untreated control showed rapid progression of tumor growth. (FIG. 7A, 7B, FIG. 14).

That is, siSHMT1 was delivered to glioblastomas in xenograft mice. SHMT1, a component of the de novo DNA biosynthetic pathway, is overexpressed during tumor growth and serves as an excellent anti-cancer target by disrupting DNA synthesis, eventually leading to tumor cell death. As an important corollary to note, other nanoparticles rely on much slower passive diffusion through the dense extracellular matrix inside the tumor and show inconsistent distribution within the tumor tissue. However, the hyperosmotic properties of X-NCs induce cell contraction, enhancing the mobility of the extracellular matrix. This allows access to hard-to-reach avascular regions inside the tumor and improves overall distribution, resulting in rapid inhibition of tumor growth by up to 97% (FIG. 7B). When X-NPs and X-NCs form complexes with nucleic acids, they are delivered in equal molar ratios, but the spatially linearly ordered configuration of X-NCs allows the drug to increase the dose concentration locally effective more rapidly and without diffusion. Therefore, X-NC not only increases the effective drug load, but also accelerates tumor growth inhibition by delivering drug molecules at high concentrations to improve the therapeutic index of drug molecules.

In subsequent experiments, protein extracts from brain tissue treated with X-NC showed an 87% reduction in SHMT1 expression compared to the control group. This is similar to the expression level of non-tumor control mice without transplantation of tumors. In addition, the X-NP treatment group showed a 65% reduction compared to the tumor control group (FIG. 7C). It clearly shows that X-NCs have more efficient and mass transport capacity due to the ordered molecules than X-NPs dispersed in equal amounts. Moreover, H&E staining suggests that X-NC does not show toxic effects on other vital organs and remaining brain tissues of mice (FIG. 7D), and safety and efficacy for in vivo application are secured.

According to the present invention, it is proved that the nanochain having high aspect ratio and high permeability can transmit materials through the BBB or BTB. A high aspect ratio effectively increases gene loading capacity.

On the other hand, the high osmotic properties of X-NC allow the BBB and BTB to open and make the exploration of solid tumors efficient. The cellular uptake mechanism was found to be related to NFAT5 function to overcome the hyperosmotic stress caused by X-NCs accessing the cell interior. These features aided X-NC-mediated siSHMT1 delivery, significantly reducing tumor volume and inhibiting further tumor growth in a xenograft brain tumor mouse model. Our strategy can provide a wide variety of anticancer drugs by using different nanochain compositions or using various gene drugs depending on the target disease. Therefore, we anticipate that this approach of ours will open a new dimension of nano medicine research to address the transfer of BBB/BTB and CNS-related treatment methods.

From the above description, researchers in the technical field to which the present invention pertains can understand that the present invention can be implemented in other specific forms without changing its technical concept or essential characteristics. In this regard, the embodiments described above should be understood as illustrative in all respects and not limiting. The scope of the present invention should be construed as including all changes or modifications derived from the meaning and scope of the claims to be described later and equivalent concepts rather than the detailed description above are included in the scope of the present invention.

1. A nucleic acid transporter of nanoplexes in a nanochain form with a linearly linked polydixylitol polymer (PdXYP) of the following Chemical Formula I.

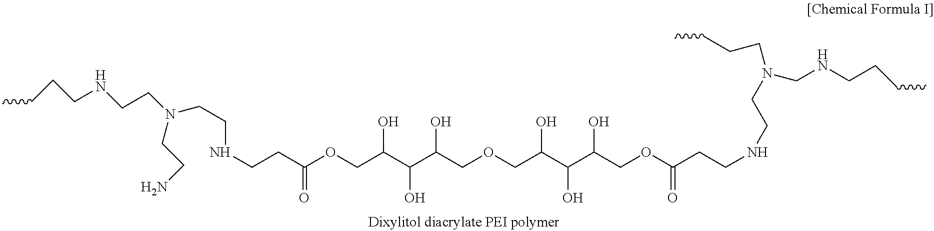

2. The nucleic acid transporter of nanoplexes according to claim 1, wherein the nucleic acid transporter is in the form of a nanochain represented by the following Chemical Formula II. 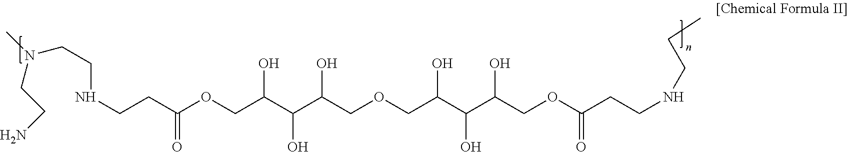
(wherein, n is an integer from 2 to 100.)

3. The nucleic acid transporter of nanoplexes according to claim 1, wherein the nucleic acid transporter is in the form of a nanochain of polydixylitol/nucleic acid nanoplexes represented by the following Chemical Formula III.

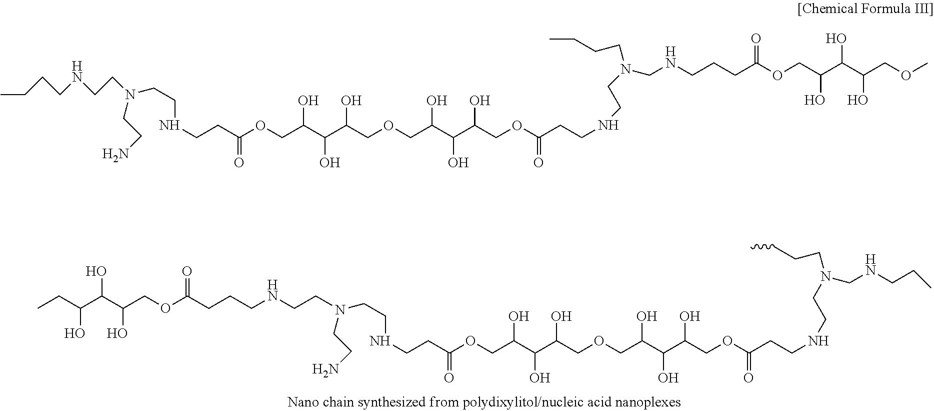

4. A nucleic acid transporter complex which is a nanocomplex in which the gene transporter according to claim 1 and a therapeutic nucleic acid form a complex.

5. The nucleic acid transporter complex according to claim 4, wherein the therapeutic nucleic acid is at least one selected from the group consisting of siRNA (small interfering RNA), shRNA (small hairpin RNA), esiRNA (endoribonuclease-prepared siRNAs), anti-sense oligonucleotide, DNA, single-stranded RNA, double-stranded RNA, DNA-RNA hybrid and ribozyme.

6. The nucleic acid transporter complex according to claim 5, wherein the therapeutic nucleic acid is SHMT1 siRNA.

7. A method for preparing a nucleic acid transporter (X-NC) in a nanochain form with a linearly linked polydixylitol polymer (PdXYP), which comprises a step of mixing the polydixylitol polymer (PdXYP) and dixylitol diacrylate (dXYdA).

8. A method for preparing a nucleic acid transporter complex, which comprises a step of mixing the nucleic acid transporter in a nanochain form (X-NC) according to claim 7 and a therapeutic nucleic acid.

9. The method for preparing a nucleic acid transporter complex according to claim 8, wherein the therapeutic nucleic acid and the nucleic acid transporter in a nanochain form (X-NC) are mixed in a molar ratio of 1:0.5 to 1:100.

10. The method for preparing a nucleic acid transporter complex according to claim 8, wherein the therapeutic nucleic acid is at least one selected from the group consisting of siRNA (small interfering RNA), shRNA (small hairpin RNA), esiRNA (endoribonuclease-prepared siRNAs), anti-sense oligonucleotide, DNA, single-stranded RNA, double-stranded RNA, DNA-RNA hybrid and ribozyme.

11. A pharmaceutical composition for gene therapy comprising the nucleic acid transporter complex according to claim 4 as an active ingredient.

12. The pharmaceutical composition for gene therapy according to claim 11, which is for cancer treatment.

13. The composition according to claim 12, wherein the cancer is selected from the group consisting of glioblastoma multiforme, lung cancer, bone cancer, pancreatic cancer, skin cancer, head and neck cancer, skin melanoma, uterine cancer, ovarian cancer, rectal cancer, colorectal cancer, colon cancer, breast cancer, uterine sarcoma, fallopian tube carcinoma, endometrium carcinoma, cervix carcinoma, vagina carcinoma, vulva carcinoma, esophageal cancer, small intestine cancer, thyroid cancer, parathyroid cancer, soft tissue sarcoma, urethral cancer, penile cancer, prostate cancer, chronic or acute leukemia, pediatric solid tumor, differentiated lymphoma, bladder cancer, kidney cancer, renal cell carcinoma, renal pelvic carcinoma, primary central nervous system lymphoma, myelencephalon tumor, brain stem glioma, and pituitary gland adenoma.
